(19) United States
(12) Patent Application Publication
(10) Pub. No.: US 2007/0244038 A1
(43) Pub. Date: Oct. 18, 2007

(54) METHODS FOR TREATING LYMPHOCYTE-ASSOCIATED DISORDERS BY MODULATION OF SIGLEC ACTIVITY

(75) Inventors: Ajit Varki, La Jolla, CA (US); Dzung Nguyen, San Diego, CA (US); Nancy Hurtado-Ziola, La Jolla, CA (US)

Correspondence Address: BUCHANAN, INGERSOLL & ROONEY LLP, P.O. BOX 1404, ALEXANDRIA, VA, 22313-1404, US

(73) Assignee: THE REGENTS OF THE UNIVERSITY OF CALIFORNIA, Oakland, CA (US)

(21) Appl. No.: 11/734,506

(22) Filed: Apr. 12, 2007

Related U.S. Application Data
(60) Provisional application No. 60/791,856, filed on Apr. 12, 2006.

(51) Int. Cl. A61K 38/16; A61K 31/739

(52) U.S. Cl. 514/8; 514/54

(57) ABSTRACT

This disclosure relates to methods for modulating lymphocyte activity and/or proliferation by regulating the activity or expression of Siglec.

### CROSS REFERENCE TO RELATED APPLICATIONS

This application claims priority to U.S. Provisional Application Ser. No. 60/791,856 filed Apr. 12, 2006, the disclosure of which is incorporated herein by reference.

### STATEMENT REGARDING FEDERALLY SPONSORED RESEARCH

This invention was supported in part by Grant No. RO1 GM32373 awarded by the National Institute of Health. The government has certain rights in this invention.

### TECHNICAL FIELD

This invention relates to modulating lymphocyte activation and proliferation by regulating the expression and activity of Sialic acid (Sia)-recognizing Ig-superfamily lectins (Siglecs).

### BACKGROUND

Siglecs are sialic acid (Sia)-recognizing Ig-superfamily lectins prominently expressed in immune cells. CD33-related-Siglecs (CD33rSiglecs, Siglecs-3 and 5-11) are a subset thought to down-regulate innate immune cell activation, via cytosolic immunoreceptor tyrosine-based inhibitory motifs (ITIMs) (Crocker & Varki, A. (2001) Trends Immunol 22:337-342; Crocker, (2005) Curr Opin Pharmacol 5:431-437; Varki & Angata, (2006) Glycobiology 16:1R-27R; and Angata et al., (2004) Proc Natl Acad Sci USA 101:13251-13256, incorporated herein by reference). These ITIMs recruit protein phosphatases, Src homology region 2 domain-containing phosphatases (SHPs), SHP-1 and SHP-2, which limit activation pathways stimulated by tyrosine kinases.

Chimpanzees and most other mammals express two major Sias at terminal ends of cell surface and secreted glycans: N-acetylneuraminic acid (Neu5Ac) and N-glycolylneuraminic acid (Neu5Gc). Human cells cannot produce Neu5Gc, due to an inactivating exon-deletion in the CMAH gene encoding the enzyme that converts CMP-Neu5Ac to CMP-Neu5Gc. The human-specific loss of Neu5Gc occurred ˜3 million years ago, and was apparently followed by rapid evolution of multiple human CD33rSiglecs, involving gene deletion, gene conversion or changes in binding specificity.

It is striking that T-cells are the only human immune cell-type that express little or no Siglecs. While all other human leukocyte types express one or more of the CD33rSiglecs at easily detectable levels, T-cells show only very low-level expression of Siglec-7 and -9. Transfection of Siglec-7 and -9 into the Jurkat T-cell line gave inhibition of T-cell receptor (TCR)-mediated signaling, indicating that CD33rSiglecs can potentially regulate T-cell activation (12). However, human T-cell expression of Siglec-7 and -9 is present only on a very small subset of CD8+ cells(Ikehara et al., (2004) J Biol Chem 279:43117-43125), and not in all individuals. With regard to human B-cells, they express some Siglecs at low levels.

As the closest evolutionary relatives, the common chimpanzees (Pan troglodytes) shares >99% identity in protein sequences with humans. Thus, it has long been assumed that chimpanzee are an effective animal model for human diseases. In fact, chimpanzee diseases may be more disparate than previously envisioned. Among the obvious differences are the lack of progression to AIDS with maintenance of CD4 T-cell counts in the great majority of chimpanzees infected with the CD4 T-cell-tropic Human Immunodeficiency Virus, and the rarity of T-cell mediated chronic active hepatitis and cirrhosis following Hepatitis B or C infection. Moreover, several other common human T-cell-mediated diseases, such as bronchial asthma, rheumatoid arthritis and type 1 diabetes have not been reported in chimpanzees or other closely related “great apes.” In some of these diseases, antibodies produced by B-cells also play a role.

Accordingly, mechanisms for modulating T and B lymphocyte activation and proliferation in humans are needed.

### SUMMARY

Provided herein are novel methods for utilizing the expression of CD33-related Siglecs (CD33rSiglecs) to limit the severity of T and B-cell-mediated pathologies. CD33rSiglec molecules contain cytosolic immunoreceptor tyrosine inhibitory motifs and have been demonstrated to inhibit activation of a variety of immune cells. In human T-cells expression of these molecules is normally very low or undetectable. However, T-cells of chimpanzee and other great apes (the closest evolutionary relatives) express multiple CD33rSiglecs. Thus, the suppression of CD33rSiglec expression in T-cells is a recent evolutionary change, relative to the ancestral condition of the ape ancestors. In keeping with this, the present data demonstrates that great ape T-cells are much less responsive to anti-TCR (CD3) or PHA stimulation. This difference can explain the increased susceptibility of humans to T-cell mediated disorders, such as HIV progression to AIDS and the late complications of viral hepatitis. Indeed, transfection of a CD33rSiglec (Siglec-5) into human T-cells makes them behave more like the great ape T-cells. The human suppression of CD33rSiglec expression in T-cells can be due to changes in gene repressors or transcription factors and/or promoter sequences. Other possibilities include epigenetic changes such as DNA methylation, chromatin modification, and siRNA action. The invention further provides methods for identifying pharmaceutical compositions that target such mechanisms and, for example, temporarily up-regulate CD33rSiglec expression on human T- or B-cells in vivo. Such induced expression of CD33rSiglecs in human T- or B-cells would limit activation and activity during acute and chronic T- or B-cell-mediated pathologies, including but not limited to such diseases as rheumatoid arthritis, asthma, inflammatory bowel disease, multiple sclerosis, psoriasis, autoimmune hepatitis, toxic shock syndrome, septic shock, type 1 diabetes, systemic lupus erythematosis, early HIV infection, infectious mononucleosis, and graft versus host disease.

The invention provides unique cell-specific methods to limit T- or B-cell activation. Current methods for T- or B-cell inhibition include non-steroidal anti-inflammatory drugs, corticosteroids, cyclosporine A, FK506, rapamycin, cyclophosphamide, statins, and anti-T-cell antibodies targeted against CD3, LFA-1, IL-2, and CD40. Most of these have serious short-term and long-term side effects, one of which includes loss of T-cells or T-cell function, resulting in serious immunosuppression. Provided herein are methods that involve inducing the expression of inhibitory proteins, which act as a signal dampening mechanism, as opposed to blocking the action of functioning proteins. The advantage is that given a strong, specific stimulus these cells could still potentially respond since their function has not been completely eliminated. The invention provides methods for applying T- or B-cell “brakes” as opposed to many of the listed drugs that essentially “kill the engine”. In addition, the normal condition of the T-cells can be restored if necessary using methods provided herein by removing the drug. The methods provided herein can preserve T- or B-cell numbers and function, while resetting their threshold for activation.

Accordingly, in one embodiment, a method for modulating lymphocyte activation is provided. The method includes contacting a lymphocyte with an agent that increases the expression and/or activity of a target Sialic acid-recognizing Ig-superfamily lectin (Siglec) associated with the lymphocyte. In some aspects the lymphocyte is a T-cell such as a CD4 or CD8 T-cell. In other aspects the lymphocyte is a B-cell.

In some embodiments the target Siglec is a CD33 related Siglec (CD33rSiglec), such as, for example, Siglec-3, Siglec-5, Siglec-6, Siglec-7, Siglec-8, Siglec-9, Siglec-10, Siglec-11, Siglec-12, Siglec-14, or other polypeptides encoded by nucleic acid sequences identified as Siglec sequences.

In some embodiments, the modulation can be by inhibition of lymphocyte activation. For example, inhibition of lymphocyte activation includes inhibition of lymphocyte proliferation. Alternatively, inhibition of lymphocyte activation may include promoting lymphocyte apoptosis.

In other embodiments, increasing the activity of a target Siglec comprises increasing the expression of endogenously-produced target Siglec. In other embodiments, increasing the activity of a target Siglec comprises expressing recombinantly-produced target Siglec. In yet another embodiment, increasing the activity of a target Siglec may include increasing stability of an endogenously-produced target Siglec.

Also provided are methods for treating a subject having, or susceptible to having, a lymphocyte-mediated pathology. The method includes administering to the subject an agent that modifies the activity of a target Siglec associated with a B-lymphocyte or T-lymphocyte. In some aspects, the agent is a compound that increases the expression of an endogenously encoded target Siglec. In other aspects, the compound may increase the half-life of a target Siglec polypeptide.

In some embodiments, the lymphocyte-mediated pathology includes rheumatoid arthritis, chronic active hepatitis, asthma, inflammatory bowel disease (IBD), multiple sclerosis (MS), psoriasis, toxic shock syndrome, HIV progression to AIDS and Systemic lupus erythematosus (SLE).

The details of one or more embodiments of the disclosure are set forth in the accompanying drawings and the description below. Other features, objects, and advantages will be apparent from the description and drawings, and from the claims.

### BRIEF DESCRIPTION OF DRAWINGS

The accompanying drawings, which are incorporated into and constitute a part of this specification, illustrate one or more embodiments of the invention and, together with the detailed description, serve to explain the principles and implementations of the invention.

FIG. 1 depicts differences in human and chimpanzee T-cells activation by PHA and anti-CD3/anti-CD28. Human and chimpanzee T lymphocytes were stimulated with soluble 10 ug/ml PHA (panel A) or with immobilized anti-CD3 (2.5 ug/ml coating conc.) plus 0.1 ug/ml soluble anti-CD28 (panel B). Cells were collected and counted on a FACSCalibur on the indicated days at 60 ul/min for 30 seconds. A log scale is used on the Y-axis to accommodate the range of values seen. Human and chimpanzee lymphocytes were labeled with anti-CD3 or anti-CD28 and detected with PE goat anti-mouse IgG (panel C). The control histogram indicates the human lymphocyte population in the presence of secondary Ab only. One representative pair out of three human and chimpanzee comparisons is presented in Panel A. The same donor pair is presented in panel B as solid symbols, along with another pair of human and chimpanzee samples (open symbols).

FIG. 2 depicts expression of CD33rSiglecs on human and great ape lymphocytes. Panel A shows percent positive lymphocytes for each Siglec antibody (staining above negative controls) for 16 chimpanzees, 5 bonobos, 3 gorillas are shown, as well as data for 8 humans (the latter were tested on one or more occasions). Examples of flow cytometry histograms of human (panel B) and chimpanzee (panel C) lymphocytes using antibodies recognizing Siglecs-3, -5, -7, and -9 (Y-axis: normalized cell numbers expressed as percent of maximum cell number detected). In later samples examined, low levels of Siglec-11 staining (<5% positive) were occasionally detected on lymphocytes in both great apes and humans. Notably, the same human studied on different days showed low or absent levels of CD33rSiglecs, indicating that the CD33rSiglec gene cluster is poised on the verge of expression.

FIG. 3 depicts anti-Siglec-5 antibodies stain chimpanzee T and B-cells. Chimpanzee lymphocytes were double-labeled with anti-Siglec-5 and PE-goat anti-mouse IgG, and with APC-anti-CD3 or APC-anti-CD19 (panel A) or with FITC-anti-CD4 and PE-Cy5-anti-CD8 (panel B). Results for CD3 and CD19 are representative of 7 individuals, and results for CD4 and CD8 are representative of 2 individuals.

FIG. 4 depicts enhanced chimpanzee T-cell response to anti-CD3 following anti-Siglec-5 antibody treatment with cross-linking, which clears Siglec-5 from the cell surface by inducing endocytosis. Human and chimpanzee T lymphocytes were stimulated with immobilized anti-CD3 plus soluble anti-CD28. For the indicated samples, anti-Siglec-5 was added at 5 ug/ml to chimpanzee lymphocytes in solution. Cells were analyzed by flow cytometry after 3 days of stimulation. Increases in forward scatter and side scatter indicate increases in cell size and internal complexity/granularity, respectively. Dead cells were excluded from analysis. Results are representative of 4 different samples from one chimpanzee.

FIG. 5 depicts human T-cell Siglec-5 expression inhibits responses to soluble anti-CD3/anti-CD28 and PHA. Unstimulated monocyte-depleted PBMCs were mock-transfected with no DNA (panel A) or transfected with 2 or 3 ug pSig5 (panel B and panel C) using the Amaxa nucleofector apparatus. After 24 h, cells were labeled with non-specific mouse IgG or anti-Siglec-5. MFI indicates the mean fluorescence intensity of Siglec-5 expression. The resulting cell populations were named control (Ctrl), Sig-5(lo), and Sig-5(hi) based on Siglec-5 expression levels. Panel D shows the results of transfected cells stimulated with soluble anti-CD3 plus soluble anti-CD28 at the indicated concentrations for 3 days. The percentages of size-expanded cells are plotted, with no stimulation background controls subtracted. Similar results were observed with a different PBMC donor. Panel E shows the results of transfected cells stimulated with 10 ug/ml of PHA for 3 days and then analyzed for CD25 expression. The histograms for CD25 staining are shown.

FIG. 6 provides bar graphs that show expression of Siglec-5 in human T-cell inhibits responses to anti-CD3/anti-CD28 beads. Panel A shows the results of unstimulated monocyte-depleted PBMCs mock transfected with no DNA or transfected with 1, 2, or 3 ug pSig5 using the Amaxa nucleofector device. After 24 h, cells were labeled with anti-Siglec-5 and goat anti-mouse IgG Alexa Fluor 488. MFI (panel A, x-axis) indicates the mean fluorescence intensity of Siglec-5 expression. Transfected cells were stimulated with anti-CD3/anti-CD28-bearing beads (see panels B, C, and D). After 3 days of stimulation, cells were analyzed for expansion in size and intracellular complexity/granularity (panel B) and increase in CD25 expression (panel C and panel D). Beads and bead-bound cells were excluded from analysis by forward scatter gating and positive auto-fluorescence of FL3.

FIG. 7 provides data indicating that Siglec-5 expression on Jurkat T-cells inhibits anti-CD3-induced intracellular calcium mobilization. Jurkat T-cells were mock transfected with no DNA or transfected with 2 ug pSig5/2×10⁶ cells using the Amaxa nucleofector device. After 24 h, Siglec-5 expression was analyzed by flow cytometry with anti-Siglec-5 and goat anti-mouse IgG Alexa Fluor 488, using a nonspecific mouse IgG as background (panel A). The percentage of Siglec-5 positive cells is indicated. Transfected cells were loaded with calcium sensing dyes, Fluo-4 and Fura Red, and then analyzed for responses to soluble anti-CD3 by real-time flow cytometric analysis (panel B). Arrow at 60 s indicates the time of mAb addition.

FIG. 8 depicts a table of various Siglec genes and chromosomal locations.

FIG. 9 provides graphs depicting the upregulation of eosinophil Siglec-F expression levels upon OVA challenge. WT mice with OVA sensitization and challenge (OVA) were compared with OVA-sensitized and PBS-challenged control mice (No OVA) for Siglec-F expression. Leukocytes from (panel A) peripheral blood, (panel B) bone marrow, or (panel C) spleen were stained with anti-CCR3 and anti-Siglec-F (or a control antibody). Cells were analyzed by flow cytometry and data plotted as the median fluorescence intensity (MFI) of anti-Siglec-F staining. Panel D depicts peripheral blood neutrophils as a control to show the specific change in eosinophil Siglec-F expression (note the different Y axis, indicating that the expression levels on neutrophils are also much lower). Histogram profiles were unimodal, making the MFI a valid means of presenting the comparisons (n=6, individual mice shown as diamonds, averages shown as bars, data shown are representative of 3 experiments). **: p<0.01.

FIG. 10 shows that Siglec-F and sialylated Siglec-F ligands are upregulated upon OVA challenge. Panel A depicts serial sections of frozen lung from WT OVA-sensitized and challenged mice were stained with antibodies against MBP (left panel, reddish brown color is positive) or Siglec-F (right panel, blue color is positive). Only the inflamed lungs were positive, as shown. Panel B depicts results using recombinant soluble Siglec-F-Fc to probe for Siglec-F ligands in the lungs from OVA-sensitized and challenged (OVA) or OVA-sensitized and PBS-challenged (No OVA) mice. Positive staining appears dark reddish-brown color. The arginine-mutated R114A Siglec-F-Fc was used as a negative control, as it is deficient in sialylated ligand binding. Results shown are typical of n=4 for each group and representative of 2 experiments. Panel C provides a higher-magnification photomicrograph of an OVA-sensitized and challenged lung section, probed with Siglec-F-Fc. Bronchiolar cells of the lung epithelia (white arrowheads) and mononuclear cells in the lung parenchyma (black arrowheads) were positive for Siglec-F ligands. Panel D shows the surface area of the Siglec-F ligand-positive bronchiolar epithelia. Mouse lungs were immunostained with Siglec-F-Fc and the area of bronchial epithelial Siglec-F-Fc immunostaining was quantitated by image analysis, with results expressed in μm²/μm length of the basement membrane of the bronchus. WT mice challenged with OVA had a significant increase in levels of Siglec-F-Fc epithelial immunostaining compared to control non-OVA challenged WT mice. Panel E shows mouse lungs immunostained with Siglec-F-Fc as above, and the number of positive peribronchial cells quantitated by image analysis. WT mice challenged with OVA had a significant increase in the numbers of peribronchial Siglec-F-Fc positive cells compared to control non-OVA challenged WT mice. ***: p<0.001.

FIG. 11 provides data indicating that Siglec-F expression is induced on activated mouse T cells in vitro and in vivo. Panel A shows spleen mononuclear leukocytes, and panel B shows peripheral blood cells. The cells were isolated and T cells activated in vitro by anti-CD3 and anti-CD28 for 3 days. Activated cells were stained by anti-Siglec-F (line) or control antibody (shaded) and analyzed by flow cytometry. Anti-CD4 or anti-CD8 were used to gate on sub-groups of T cells. Panel C shows ung sections from chronically OVA-challenged WT mice stained with anti-CD4 and anti-Siglec-F antibodies.

FIG. 12 provides data indicating Siglec-F−/− mice have elevated eosinophilic inflammation in lung, peripheral blood and bone morrow in an OVA-induced lung allergy model. WT or Siglec-F−/− mice were either OVA-sensitized and challenged (OVA), or OVA-sensitized and PBS-challenged (No OVA). All groups were compared for numbers of eosinophils in airway (panel A and panel B), blood (panel C), and bone marrow (panel D) (n=6 mice/group, data shown is representative of three experiments). Panel A depicts WT and Siglec-F−/− OVA lung sections stained for MBP. Dark red stained peribronchial MBP+ cells were counted as eosinophils, and 8-10 bronchi/slide were counted. Panel B provides quantitative results derived from panel A, expressed as the number of eosinophils per bronchus. Panel C shows peripheral blood leukocytes and panel D shows bone marrow cells stained with Wright-Giemsa and differential cell counts taken under a light microscope. *: p<0.05, **: p<0.01, ***: p<0.001.

FIG. 13 provides data indicating that eosinophil resolution after OVA challenge is delayed in Siglec-F−/− mice and peribronchial cell apoptosis is decreased. Panel A shows a cell count from mice euthanized 7 days after the last OVA challenge. Eosinophils/bronchus were enumerated, as in FIG. 4. Panel shows counted eosinophils in the BAL. Panel C shows a cell count from lung sections stained for apoptotic cells by TUNEL assay (n=4, data representative of two experiments). *: p<0.05, ***: p<0.001.

Like reference symbols in the various drawings indicate like elements.

### DETAILED DESCRIPTION

The invention provides the first report of a disparity between humans and chimpanzees in T-cell activation via the TCR and the correlative expression of the inhibitory CD33rSiglec molecules only in great ape T-cells. In one exemplary embodiment, the information provided herein demonstrate an inhibitory role for Siglec-5 on chimpanzee T-cells and show that induced expression of human Siglec-5 in human T-cells mimics the chimpanzee phenotype.

In general, provided herein are methods for modulating T-lymphocyte activation and/or proliferation by regulating Siglec expression and activity. In some embodiments the method includes inducing human T-cell Siglec expression for the purpose of limiting T-cell activation and responsiveness. The method may include using a pharmaceutical composition or natural product to provide induction. Siglec molecules contain inhibitory motifs that are known to limit T-cell signaling pathways. Therefore, inducing Siglec expression is likely to inhibit T-cell activation without causing cell death. Also provided are methods for screening for small molecules that would up-regulate CD33rSiglec expression on human T-cells. In addition, methods for permanently activating expression of CD33rSiglecs, such as gene therapy, are included.

Unless defined otherwise, all technical and scientific terms used herein have the same meaning as commonly understood by one of ordinary skill in the art to which the invention pertains. Although any methods and materials similar or equivalent to those described herein can be used in the practice for testing of the invention, the preferred materials and methods are described herein. In describing and claiming the invention, the following terminology will be used.

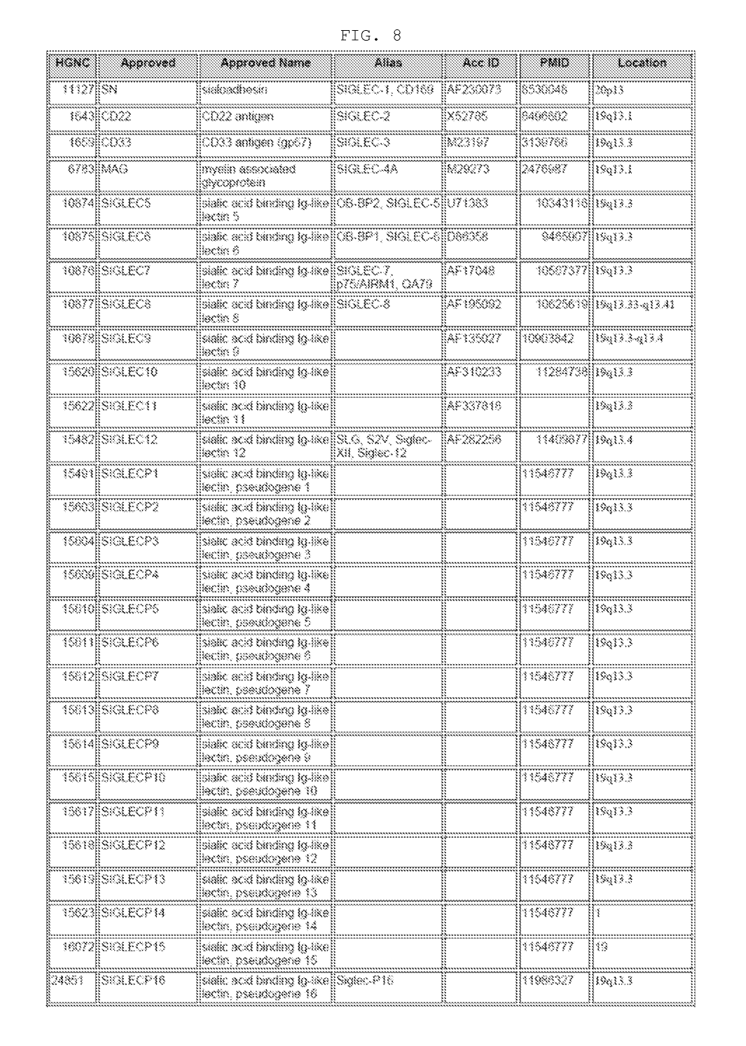

As used herein, the term “Siglecs” means a sialic acid binding Ig-like lectin. Exemplary Siglecs include Siglec-3—CD33, I-type lectin from myeloid progenitors mature monocytes; Siglec-5—I-type lectin from monocytes, neutrophils; Siglec-6—OB-BP1, I-type lectin from B-cells, placental trophoblasts; Siglec-7—AIRM1, I-type lectin from NK cells, monocytes; Siglec-8—SAF-2, I-type lectin from eosinophils, mast T-cells; Siglec-9—I-type lectin from monocytes, neutrophils, NK cells (subset); Siglec-10—I-type lectin from B-cells, eosinophils, monocytes; and Siglec-11—I-type lectin; Siglec-12, I-type lectin; or Siglec-14, I-type lectin. The location of the genes encoding such Siglecs is included in FIG. 8.

Also as used herein, the term “ITIMS” means Immunoreceptor Tyrosine-based Inhibitory Motifs. The term “mAb” means monoclonal antibody. The term “Sia” means sialic acid. The term “Neu5Ac” means N-acetylneuraminic acid. The term “Neu5Gc” means N-glycolylneuraminic acid. The term “TCR” means T-cell receptor.

“Test compound” or “agent” refers to any compound tested as a modulator of Siglec expression and/or activation. The test compound can be any small organic molecule, or a biological entity, such as a protein, e.g., an antibody or peptide, a sugar, a nucleic acid, e.g., an antisense oligonucleotide, RNAi, or a ribozyme, or a lipid. Alternatively, test compound can be modulators that are genetically altered versions of a target Siglec polypeptide. Typically, test compounds will be nucleic acids, small organic molecules, peptides, lipids, or lipid analogs.

Over-activation of a subjects immune system can result in an autoimmune disease. “Autoimmune disease” refers to a disease caused by an inability of the immune system to distinguish foreign molecules from self molecules, and a loss of immunological tolerance to self antigens, that results in destruction of the self molecules. Autoimmune diseases, include but are not limited to, insulin-dependent diabetes mellitus (IDDM), multiple sclerosis, experimental autoimmune encephalomyelitis (an animal model of multiple sclerosis), rheumatoid arthritis, experimental autoimmune arthritis, myasthenia gravis, thyroiditis, an experimental form of uveoretinitis, Hashimoto's thyroiditis, primary myxoedema, thyrotoxicosis, pernicious anaemia, autoimmune atrophic gastritis, Addison's disease, premature menopause, male infertility, juvenile diabetes, Goodpasture's syndrome, pemphigus vulgaris, pemphigoid, sympathetic ophthalmia, phacogenic uveitis, autoimmune haemolytic anaemia, idiopathic leucopenia, primary biliary cirrhosis, active chronic hepatitis Hb.sub.s-ve, cryptogenic cirrhosis, ulcerative colitis, Sjogren's syndrome, scleroderma, Wegener's granulomatosis, Poly/Dermatomyositis, discoid LE and systemic Lupus erythematosus.

“Immune cell response” refers to the response of immune system cells to external or internal stimuli (e.g., antigen, cytokines, chemokines, and other cells) producing biochemical changes in the immune cells that result in immune cell migration, killing of target cells, phagocytosis, production of antibodies, other soluble effectors of the immune response, and the like.

“T-lymphocyte response” and “T-lymphocyte activity” are used here interchangeably to refer to the component of immune response dependent on T-lymphocytes (i.e., the proliferation and/or differentiation of T-lymphocytes into helper, cytotoxic killer, or suppressor T-lymphocytes, the provision of signals by helper T-lymphocytes to B-lymphocytes that cause or prevent antibody production, the killing of specific target cells by cytotoxic T-lymphocytes, and the release of soluble factors such as cytokines that modulate the function of other immune cells).

“Immune response” refers to the concerted action of lymphocytes, antigen presenting cells, phagocytic cells, granulocytes, and soluble macromolecules produced by the above cells or the liver (including antibodies, cytokines, and complement) that results in selective damage to, destruction of, or elimination from the human body of invading pathogens, cells or tissues infected with pathogens, cancerous cells, or, in cases of autoimmunity or pathological inflammation, normal human cells or tissues.

“Patient”, “subject” or “mammal” are used interchangeably and refer to mammals such as human patients and non-human primates, as well as experimental animals such as rabbits, rats, and mice, and other animals. Animals include all vertebrates, e.g., mammals and non-mammals, such as sheep, dogs, cows, chickens, amphibians, and reptiles.

“Treating” or “treatment” includes the administration of the compositions, compounds or agents of the invention to prevent or delay the onset of the symptoms, complications, or biochemical indicia of a disease, alleviating or ameliorating the symptoms or arresting or inhibiting further development of the disease, condition, or disorder (e.g., a disease or condition that is a result of immune system over-activation). “Treating” further refers to any indicia of success in the treatment or amelioration or prevention of the disease, condition, or disorder, including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms can be based on objective or subjective parameters; including the results of an examination by a physician. Accordingly, the term “treating” includes the administration of the compounds or agents of the invention to prevent or delay, to alleviate, or to arrest or inhibit development of the symptoms or conditions associated with immune system over-activation. The term “therapeutic effect” refers to the reduction, elimination, or prevention of the disease, symptoms of the disease, or side effects of the disease in the subject. “Treating” or “treatment” using the methods of the invention includes preventing the onset of symptoms in a subject that can be at increased risk of immune system over-activation but does not yet experience or exhibit symptoms, inhibiting the symptoms of immune system over-activation (slowing or arresting its development), providing relief from the symptoms or side-effects of the condition, and relieving the symptoms of the condition (causing regression). Treatment can be prophylactic (to prevent or delay the onset of the disease, or to prevent the manifestation of clinical or subclinical symptoms thereof) or therapeutic suppression or alleviation of symptoms after the manifestation of the disease or condition.

“Activators,” and “modulators” of Siglec expression and/or activity in cells are used to refer to activating or modulating molecules, respectively, identified using in vitro and in vivo assays for agents that modulate Siglec expression and/or activity, e.g., ligands, agonists, antagonists, and their homologs and mimetics.

“Modulator” includes activators. Activators are agents that, e.g., bind to, stimulate, increase, open, activate, facilitate, enhance activation, sensitize or up regulate the activity of a target Siglec, e.g., agonists. Modulators include agents that, e.g., alter the expression and/or activity of a target Siglec with: nucleic acids (e.g., DNA, RNA, siRNA, antisense RNA), small molecules, proteins that bind activators or inhibitors, receptors, including proteins, peptides, lipids, carbohydrates, polysaccharides, or combinations of the above, e.g., lipoproteins, glycoproteins, and the like.

Provided herein are methods for modulating the activity of a lymphocyte by regulating the activity of a target Siglec. As used herein, “regulating the activity of a target Siglec” includes: 1) mechanisms for activating endogenous nucleic acid sequences that encode a target Siglec such that Siglec polypetide levels are increased in a cell; 2) introducing exogenous nucleic acid sequences encoding a target Siglec in to a cell such that Siglec polypeptide levels are increased in a cell; 3) reducing the turnover rate of endogenous Siglec polypeptides such that Siglec polypetide levels are increased in a cell.

In some embodiments the methods described herein can be designed to identify substances that modulate the biological activity of a Siglec by affecting the expression of a Siglec nucleic acid sequence encoding a Siglec polypeptide. For example, methods can be utilized to identify compounds that bind to Siglec regulatory sequences. Alternatively, methods can be designed to identify substances that modulate the biological activity of a Siglec by affecting the half-life of a Siglec polypeptide.

In other embodiments, methods for treating a Siglec-related condition by methods of the invention are provided. Siglec polypeptides, nucleic acid sequences encoding a Siglec polypeptide, substances or compounds that regulate the expression of endogenous Siglecs or the half-life of endogenous Siglecs may be used for modulating the activity of a lymphocyte. In general these methods can be used in the treatment of conditions associated with disorders related to over-activation of lymphocytes.

Disorders and diseases treatable by the methods and compositions of the invention include, but are not limited to: rheumatologic disorders (e.g., rheumatoid arthritis, psoriatic arthritis, seronegative spondyloarthropathies), bone marrow or solid organ transplant, graft-versus-host reaction, inflammatory conditions, autoimmune disorders (e.g., systemic lupus erythematosus, Hashimoto's thyroiditis, Sjogren's syndrome), allergies (e.g., asthma, allergic rhinitis), neurologic disorders (e.g., Alzheimer's, Parkinson's, dementia, brain cancer, Bell's palsy, post-herpetic neuralgia), cancers (e.g., lymphoma, B-cell, T-cell and myeloid cell leukemias), infections (e.g., bacterial, parasitic, protozoal and viral infections, including AIDS), chemotherapy or radiation-induced toxicity, cachexia, cardiovascular disorders (e.g., congestive heart failure, myocardial infarction, ischemia/reperfusion injury, arteritis, stroke), diabetes mellitus, skin diseases (e.g., psoriasis, scleroderma, dermatomyositis), hematologic disorders (e.g., myelodysplastic syndromes, acquired or Fanconi's aplastic anemia), septic shock, liver diseases (e.g., viral hepatitis or alcohol-associated), bone disorders (e.g., osteoporosis, osteopetrosis).

Human T-cells give much stronger proliferative responses to specific activation via the T-cell receptor (TCR), compared to those from chimpanzees, the closest evolutionary relatives. Non-specific activation using phytohemagglutinin (PHA) was robust in chimpanzee T-cells, indicating that the much lower response to TCR simulation is not due to any intrinsic inability to respond to an activating stimulus. CD33-related-Siglecs are inhibitory signaling molecules expressed on most immune cells, and are thought to downregulate cellular activation pathways via cytosolic immunoreceptor tyrosine-based inhibitory motifs. Among human immune cells, T lymphocytes are a striking exception, expressing little to none of these molecules. In stark contrast, the present studies indicate that T lymphocytes from chimpanzees as well as the other closely related “great apes” (bonobos, gorillas, and orangutans) express several CD33-related-Siglecs on their surfaces. Thus, human-specific loss of T-cell Siglec expression occurred after the last common ancestor with great apes, potentially resulting in an evolutionary difference, with regard to inhibitory signaling. The present studies have conformed this finding by investigating Siglec-5, which is prominently expressed on chimpanzee lymphocytes, including CD4 T-cells. Antibody-mediated clearance of Siglec-5 from chimpanzee T-cells enhanced TCR-mediated activation. Conversely, primary human T-cells and Jurkat T-cells transfected with Siglec-5 become less responsive i.e., they behave more like chimpanzee T-cells.

The low but variable expression of CD33rSiglecs on human T-cells suggests that they are “poised” to be induced for high expression. Accordingly, provided herein is a novel model to explain differences in human and chimpanzee T-cell stimulation. The data indicates that these differences contribute to the involvement of T-cells in human diseases, particularly AIDS and chronic active hepatitis.

Modulating enhancement of Siglec expression and/or activity as a means of down-regulating an immune response in a subject is useful in therapy. An individual having a condition which involves or is precipitated by an overactive immune response would benefit from the down-regulation of that immune response. Down-regulation of immune responses can be in the form of up-regulating Siglec expression, such as Siglec-5, on a lymphocyte. To achieve treatment of such an individual, any agent that up-regulates expression and/or activity of a target Siglec can be used. The agent can be in the form of a small molecule that modulates Siglec expression and/or activity in an individual. Alternatively, in vivo gene therapy can be used to activate nucleic acid sequences associated with Siglec expression in a lymphocyte. Moreover, ex vivo therapy can be used to introduce a nucleic acid molecule in to the cells of a subject such that the cells will express or over-express a target Siglec.

For example, in one embodiment, administration of an agent that promotes enhancement of Siglec expression and/or activity is therapeutically useful in situations where down-regulation of antibody and cell-mediated responses would be beneficial. In certain instances, it may be desirable to further administer other agents that down-regulate immune responses in order to further limit the immune response. Alternatively, immune responses can be down-regulated in a subject by removing immune cells from the subject and deactivating the immune cells by methods which include contacting the immune cells with an agent that promotes Siglec expression and/or activity and reintroducing the in vitro deactivated immune cells into the subject. Accordingly, immune cells can be obtained from a subject and cultured and inactivated or deactivated ex vivo in the presence of an agent that promotes Siglec expression and/or activity. The population of ex vivo cells can be expanded and then administered to a subject.

For administration to a subject, modulators of Siglec expression and/or activity (e.g., stimulatory agents, nucleic acid molecules, proteins, or compounds identified as modulators of a Siglec expression and/or activity) will preferably be incorporated into pharmaceutical compositions suitable for administration. Such compositions typically comprise the nucleic acid molecule, protein, or compound and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL.™. (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it is preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

In one embodiment, modulatory agents are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations should be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose can be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma can be measured, for example, by high performance liquid chromatography.

A method for identifying a modulator of Siglec expression and/or activity is provided which includes contacting a test compound (e.g., an agent) with a cell-based assay system comprising a cell capable of expressing a target Siglec, providing the test compound to the assay system in an amount selected to be effective to enhance Siglec expression and/or activity, and detecting an effect of the test compound on Siglec expression and/or activation in the assay system, effectiveness of the test compound in the assay being indicative of the modulation.

The invention also pertains to the field of predictive medicine in which diagnostic assays, prognostic assays, pharmacogenomics, and monitoring clinical trails are used for prognostic (predictive) purposes to thereby treat an individual prophylactically. As such, the invention contemplates use of methods provided herein to screen, diagnose, stage, prevent and/or treat disorders characterized by under expression or activity of a target Siglec. Accordingly, a subject can be screened to determine the level of a particular Siglec's expression or activity. A subject can also be screened for the susceptibility of immune cells to techniques that enhance the expression or over expression of a target Siglec.

Thus, various aspects of the invention relates to diagnostic assays for determining expression of a Siglec, in the context of a biological sample (e.g., blood, serum, cells, tissue).

The invention also provides for prognostic (or predictive) assays for determining whether an individual is at risk of developing a disorder associated with under expression of a target Siglec. Such assays can be used for prognostic or predictive purpose to thereby prophylactically treat an individual prior to the onset of a disorder characterized by or associated with under expression or activity of a target Siglec.

As previously noted, the invention encompasses agents which modulate expression or activity of a Siglec. An agent may, for example, be a small molecule. For example, such small molecules include, but are not limited to, peptides, peptidomimetics, amino acids, amino acid analogs, polynucleotides, polynucleotide analogs, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds. The factors to consider in choosing an appropriate dose of a small molecule agent will be understood by the ordinarily skilled physician, veterinarian, or scientist. The dose(s) of the small molecule will vary, for example, depending upon the identity, size, and condition of the subject or sample being treated, further depending upon the route by which the composition is to be administered, if applicable, and the effect which the practitioner desires the small molecule to have upon the polynucleotide or polypeptide of the invention. Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram. It is furthermore understood that appropriate doses of a small molecule depend upon the potency of the small molecule with respect to the expression or activity to be modulated. Such appropriate doses may be determined using the assays described herein. When one or more of these small molecules is to be administered to an animal (e.g., a human) in order to modulate expression or activity of a polypeptide or polynucleotide of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, polynucleotideral health, gender, and diet of the subject, the time of administration, the route of administration, the rate of excretion, any drug combination, and the degree of expression or activity to be modulated.

High throughput screening methodologies are particularly envisioned for the detection of modulators of a target Siglec, such as Siglec-5, described herein. Such high throughput screening methods typically involve providing a combinatorial chemical or peptide library containing a large number of potential therapeutic compounds (e.g., ligand or modulator compounds). Such combinatorial chemical libraries or ligand libraries are then screened in one or more assays to identify those library members (e.g., particular chemical species or subclasses) that display a desired characteristic activity. The compounds so identified can serve as conventional lead compounds, or can themselves be used as potential or actual therapeutics.

A combinatorial chemical library is a collection of diverse chemical compounds generated either by chemical synthesis or biological synthesis, by combining a number of chemical building blocks (i.e., reagents such as amino acids). As an example, a linear combinatorial library, e.g., a polypeptide or peptide library, is formed by combining a set of chemical building blocks in every possible way for a given compound length (i.e., the number of amino acids in a polypeptide or peptide compound). Millions of chemical compounds can be synthesized through such combinatorial mixing of chemical building blocks.

The preparation and screening of combinatorial chemical libraries is well known to those having skill in the pertinent art. Combinatorial libraries include, without limitation, peptide libraries (e.g. U.S. Pat. No. 5,010,175; Furka, 1991, Int. J. Pept. Prot. Res., 37:487-493; and Houghton et al., 1991, Nature, 354:84-88). Other chemistries for generating chemical diversity libraries can also be used. Nonlimiting examples of chemical diversity library chemistries include, peptoids (PCT Publication No. WO 91/019735), encoded peptides (PCT Publication No. WO 93/20242), random bio-oligomers (PCT Publication No. WO 92/00091), benzodiazepines (U.S. Pat. No. 5,288,514), diversomers such as hydantoins, benzodiazepines and dipeptides (Hobbs et al., 1993, Proc. Natl. Acad. Sci. USA, 90:6909-6913), vinylogous polypeptides (Hagihara et al., 1992, J. Amer. Chem. Soc., 114:6568), nonpeptidal peptidomimetics with glucose scaffolding (Hirschmann et al., 1992, J. Amer. Chem. Soc., 114:9217-9218), analogous organic synthesis of small compound libraries (Chen et al., 1994, J. Amer. Chem. Soc., 116:2661), oligocarbamates (Cho et al., 1993, Science, 261:1303), and/or peptidyl phosphonates (Campbell et al., 1994, J. Org. Chem., 59:658), nucleic acid libraries (see Ausubel, Berger and Sambrook, all supra), peptide nucleic acid libraries (U.S. Pat. No. 5,539,083), antibody libraries (e.g., Vaughn et al., 1996, Nature Biotechnology, 14(3):309-314) and PCT/US96/10287), carbohydrate libraries (e.g., Liang et al., 1996, Science, 274-1520-1522) and U.S. Pat. No. 5,593,853), small organic molecule libraries (e.g., benzodiazepines, Baum C&EN, Jan. 18, 1993, page 33; and U.S. Pat. No. 5,288,514; isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; and the like).

United States Patent Application Publication No. 20040176309, 20020110862, 20030036831, and 20030040604 are hereby incorporated by reference, in their entirety for all purposes. While these publications provide general information about Siglecs, it is understood that they do not propose or describe the methods provided herein.

### EXAMPLE 1

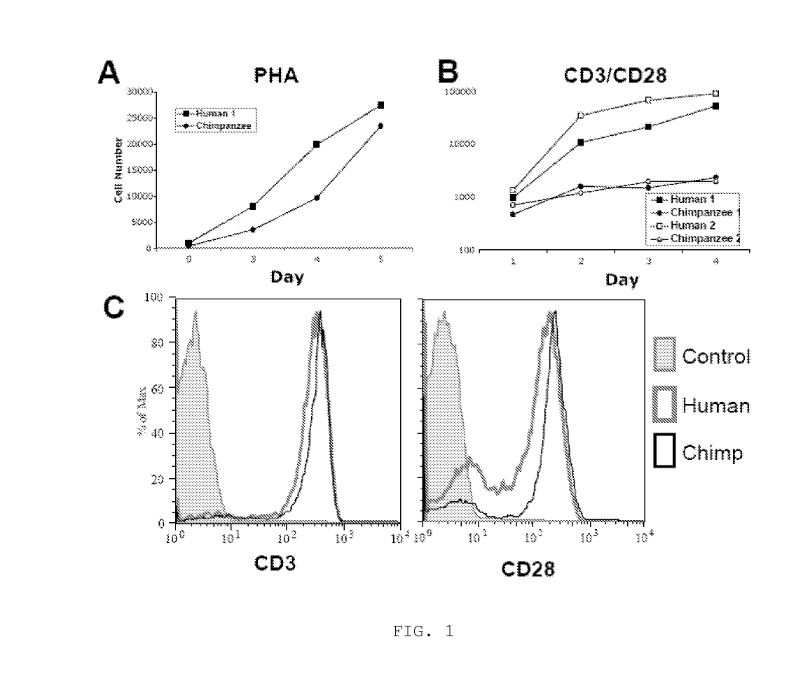

Chimpanzee T-cells are much less responsive to TCR stimulation than human T-cells. The general responsiveness of freshly isolated human and chimpanzee T-cells was evaluated by activation with the lectin phytohaemagglutinin-L (PHA), which non-specifically stimulates T-cells by random cross-linking of surface proteins. Both cell types responded robustly, with the proliferation of chimpanzee cells being somewhat lower (FIG. 1, panel A). This data is consistent with previous studies indicating that responses of chimp T-cells to some superantigens were as robust as responses by human T-cells. In the present studies human and chimpanzee T-cell activation was examined by TCR activation using immobilized anti-CD3 along with co-stimulation by soluble anti-CD28. Under these more physiological conditions, chimpanzee T-cells proliferated much less than human T-cells (FIG. 1, panel B). After 5 days of activation, chimpanzee T-cell numbers were about two orders of magnitude lower than in humans. No major differences in CD3 or CD28 levels on human and chimpanzee T-cells could account for this (FIG. 1, panel C). Thus, while chimpanzee T-cells can proliferate upon non-specific lectin-mediated activation, there is a striking disparity with human T-cells following physiologically relevant activation via the TCR.

Great Apes express higher levels and wider varieties of CD33rSiglecs on lymphocytes in comparison to humans. Siglec expression on immune cells of great apes and other nonhuman primates has not been previously studied. CD33rSiglec expression on lymphocytes from humans was compared with all four great ape species (chimpanzees, bonobos, gorillas, and orangutans) using previously characterized mAbs against human Siglecs-3, and -5 thru -11. Given the very close genetic similarity of humans and great apes, most or all of the mAbs were expected to cross-react. Indeed, the present studies show that all bound recombinant Siglec human and chimpanzee CD33rSiglecs bind equally well in ELISA assays.

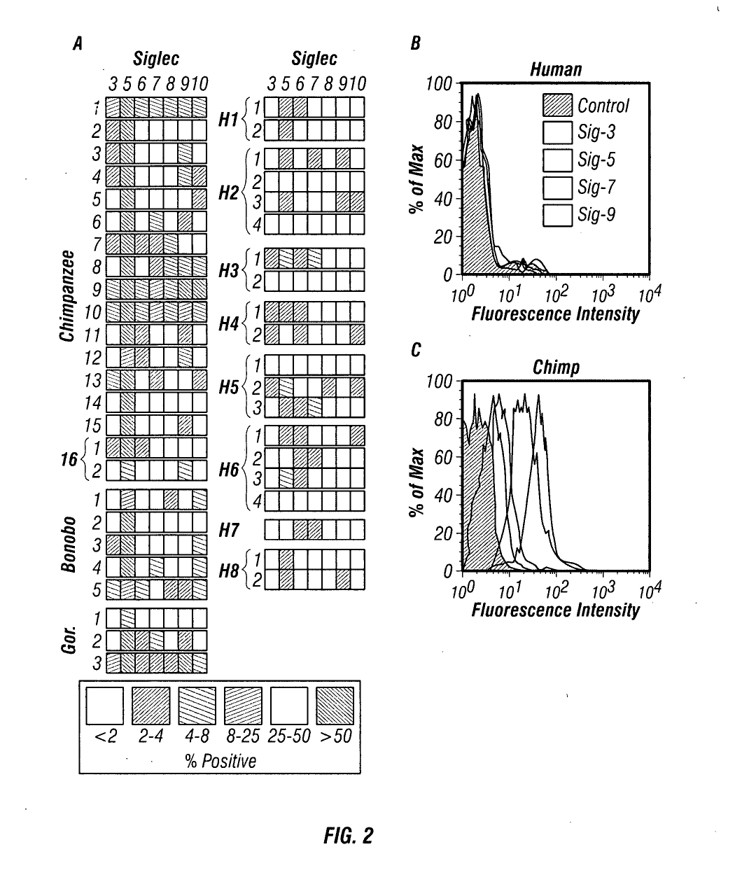

Differences in CD33rSiglec expression between human and great ape lymphocytes were identified. Anti-Siglec-5 staining was consistently found in all chimpanzees studied, and several other CD33rSiglecs were variably expressed (FIG. 2). Positive staining for anti-Siglec-5 ranged from 11 to 98% of total chimpanzee lymphocytes. In contrast, humans representing a range of geographic and ethnic origins demonstrated very weak and transient expression of CD33rSiglecs on lymphocytes (8 humans are shown, representative of 16 tested). One human subject showed Siglec-7 expression in 34% of lymphocytes, but upon re-testing, no significant expression was observed (<2% positive). This could reflect changes in the percent of NK cells, which are known to be Siglec-7 positive and can vary in number. In most humans, expression of any CD33rSiglec rarely exceeded 4 percent of lymphocytes. Interestingly, chimpanzee #16 was analyzed on two separate occasions and also demonstrated some variability in Siglec expression (FIG. 2, panel A). Overall, while 19 chimpanzee samples showed an average of ˜60% anti-Siglec-5 positive lymphocytes, the average for 28 human samples was 3.4%. Outgroup comparisons revealed a similar expression of multiple CD33rSiglecs (particularly Siglec-5) on lymphocytes of bonobos and gorillas (FIG. 2, panel A). Several other CD33rSiglecs also showed significant and variable expression on great ape, but not human lymphocytes (FIG. 2, panel A). One orangutan sample demonstrated relatively high expression of Siglec-6 (41% positive) but lower expression of Siglecs-3, -5, -7, and -10 (13%, 7%, 5%, and 18% positive, respectively). The results for orangutan expression of Siglec-5 are inconclusive due to low detection on monocytes and granulocytes, which normally express high levels of Siglec-5 in humans and great apes. This may be due to poor mAb recognition of Siglec-5 in this great ape species that is most distantly related to humans.

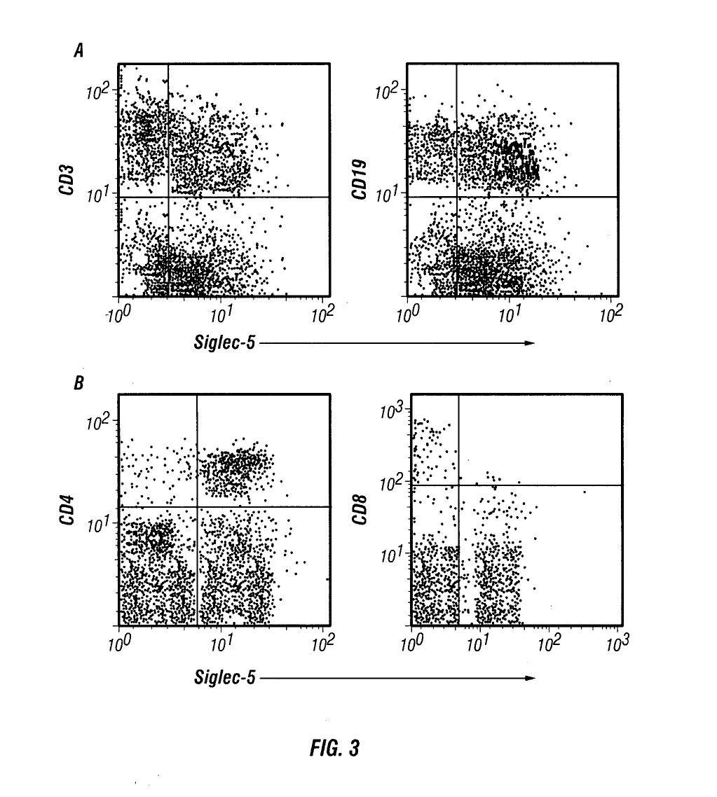

Siglec-5 is expressed on chimpanzee, but not human, B-cells and CD4+ T-cells. Further characterization of Siglec-5+ lymphocytes from 7 chimpanzees revealed expression on CD3+ T-cells as well as CD19+ B-cells (see FIG. 3, panel A for representative results). Double-staining flow cytometric analysis of chimpanzee lymphocytes revealed that the majority of CD4+ T-cells expressed Siglec-5 (FIG. 3, panel B, 83%). In contrast, only 5% of CD8+ T-cells were positive for Siglec-5. Corroborating the earlier findings, both CD4 and CD8 T-cells in humans were negative for Siglec-5 (<2% positive).

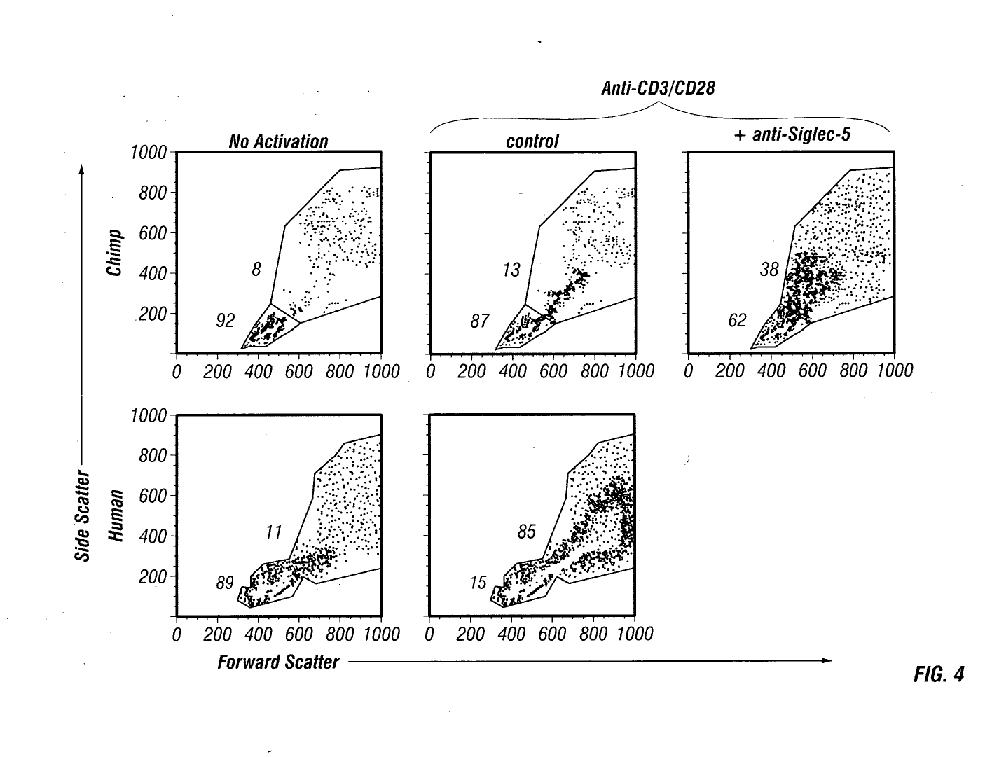

Antibody-induced Siglec-5 internalization partially releases inhibition of chimpanzee T-cell stimulation. Chimpanzee Siglec-5 contains a mutation that renders it potentially unable to bind Sias compared to the human Siglec-5 orthologue (4). While this could alter Siglec-5 signaling, similar mutations in Siglec-2 and Siglec-9 do not completely abolish inhibitory function (12, 26). Thus, the prominent expression of Siglec-5 on chimpanzee lymphocytes is predicted to inhibit TCR/CD3-mediated activation signals. To address this, chimpanzee cells were studied in the presence or absence of soluble anti-Siglec-5 mAbs during stimulation with immobilized anti-CD3. Anti-Siglec-5 mAbs induced 40 to 70% internalization of cell surface Siglec-5 after 1 h at 37° C. while not affecting CD3 levels. After 3 days of incubation a significant increase in expanded cells was identified, as evidenced by increases in flow cytometric side and forward scatter (FIG. 4). Although this approach did not increase chimpanzee T-cell proliferation to the level seen with humans, the results indicate that Siglec-5 can contribute significantly to regulating the TCR-initiated response in chimpanzee cells.

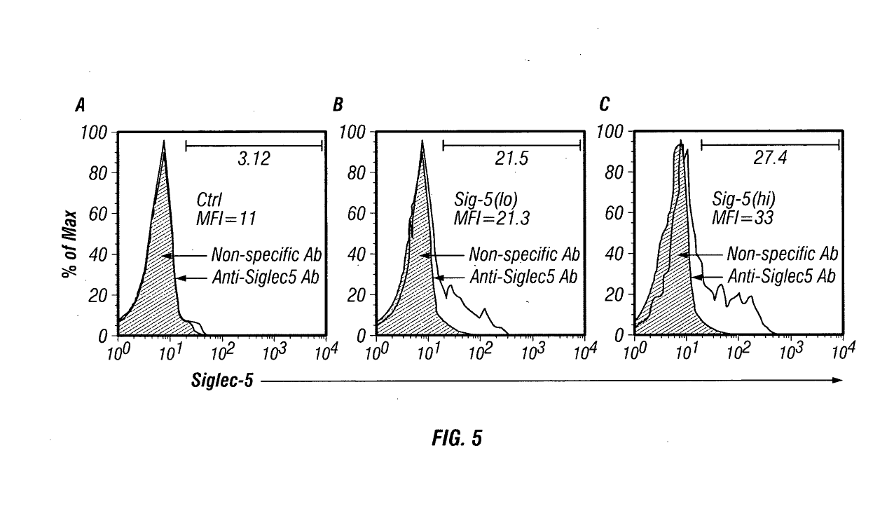

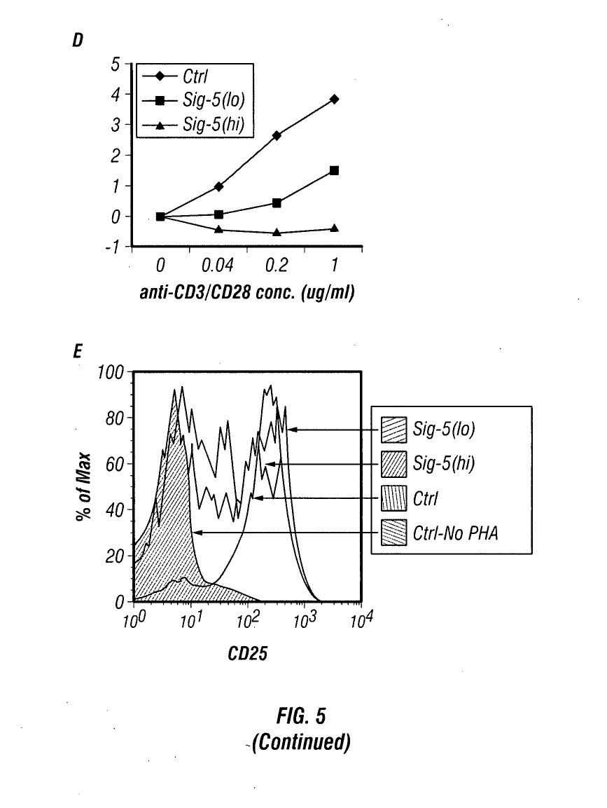

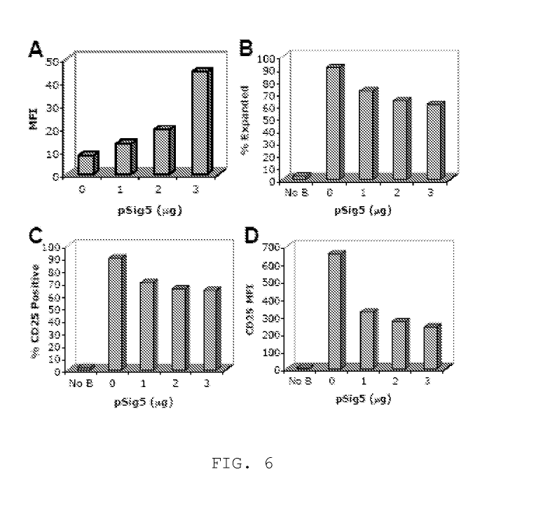

Induced expression of Siglec-5 in primary human T-cells inhibits TCR responses. The present studies further determined whether induced expression of human Siglec-5 in human T-cells modulated proliferation. Using Amaxa nucleofection, Siglec-5 expression was induced in resting human T-cells (FIGS. 5 and 6). In one experiment, monocyte-depleted PBMCs were nucleofected with 0, 2, or 3 μg of a plasmid construct containing full length human Siglec-5 (pSig5). The resulting subpopulations were designated as control (Siglec-5-), Sig-5(lo), or Sig-5(hi) based upon relative expression of Siglec-5 (FIG. 5, panels A-C). All three populations demonstrated no significant changes in forward scatter, side scatter or expression of CD4, suggesting a uniform resting state for each population. Twenty-four hours after nucleofection, immobilized anti-CD3 and soluble anti-CD28 mAbs were added at varying concentrations. After three days, significant inhibitory effects on cell proliferation were observed in a Siglec-5 expression-dependent manner (FIG. 5, panel D). With background size-expanded cells normalized to zero in the absence of anti-CD3/anti-CD28, there was no increase in size-expanded cells for high Siglec-5 expressing cells, and a decreased number of size-expanded cells compared to the control at all mAb concentrations (FIG. 5, panel D). The same three cell populations were also stimulated with PHA for three days. In contrast to mAb stimulation, a larger percentage of cells in all three populations were activated by PHA, as measured by CD25 expression (FIG. 5, panel E). Sig-5(lo) and Sig-5(hi) cells responded less robustly than control cells, as quantitated by percent of cells expanded (50%, 46%, and 86% respectively) and mean fluorescence intensity of CD25 (50, 24, and 244 respectively). These results correlate well with the differences observed between human and chimpanzee T-cells, based on Siglec-5 expression.

A different stimulation method using anti-CD3/anti-CD28-coated beads (DynabeadsO CD3/CD28 T-cell Expander) to stimulate Siglec-5 expressing cells was employed. Nucleofecting primary lymphocytes with 0, 1, 2, and 3 μg of pSig5 produced Siglec-5 expressing cells in a dose-dependent manner (FIG. 6, panel A). After 5 days of incubation at a cell to bead ratio of 1:1, Siglec-5 expression-dependent inhibition of stimulation was observed as measured by percentage of expanded cells (FIG. 6, panel B), percent of cells expressing CD25 (FIG. 6, panel C), and the mean fluorescence intensity of CD25 of each cell population (FIG. 6, panel D). These results further indicate that Siglec-5 can inhibit anti-CD3/anti-CD28 responsiveness in T-cells. To confirm whether Siglec-5 expressing cells were truly the “non-responding” cell, expanded and non-expanded cells were gated after CD3/CD28-bead stimulation and stained for Siglec-5 in each subpopulation. Non-expanded cells demonstrated a higher percentage of cells positive for Siglec-5 than expanded cells, for all three transfected populations. Thus, Siglec-5 expressing cells are less responsive and Siglec-5 negative cells are more likely to respond given the same stimulation.

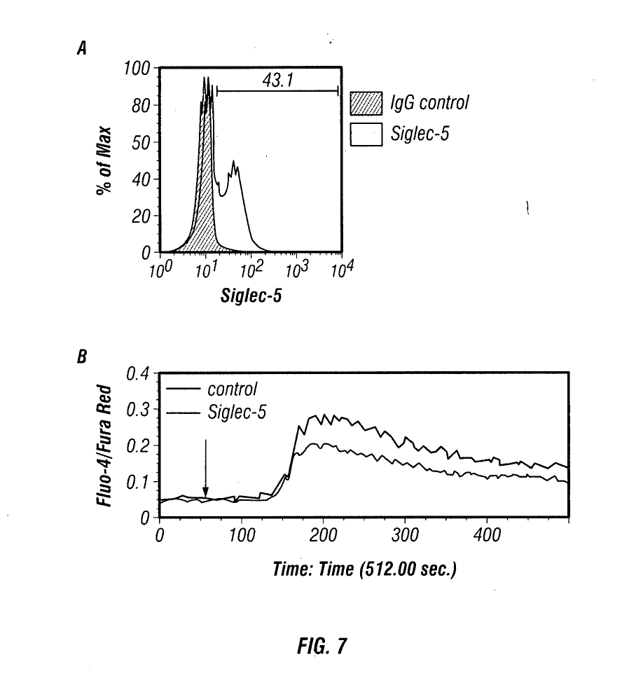

Expression of Siglec-5 in Jurkat T-cells inhibits anti-CD3-induced intracellular calcium mobilization. To measure more proximate effects of Siglec-5 expression on CD3 stimulation, intracellular calcium mobilization assays were performed on transfected and mock transfected control Jurkat T leukemia cells. Using Amaxa nucleofection, Siglec-5 was transiently expressed in up to 43% of cells 24 h after nucleofection (FIG. 7, panel A). Subsequent intracellular calcium mobilization in response to anti-CD3 mAb was reduced compared to controls, using real-time flow cytometric calcium measurements (FIG. 7, panel B). The inhibitory effects were consistent in 3 separate experiments. These data further suggest that CD3 activation is regulated by Siglec-5 at the level of signal initiation that leads to calcium flux.

Mechanism of Down-regulation of Siglec-5 on Human T-cells. To explore the mechanism of down-regulation, human T-cells were studied for staining by anti-Siglec-5 mAbs, without or with membrane permeabilization, which would allow the mAbs to access intracellular compartments. A low level of Siglec-5 staining was identified in human lymphocytes following permeabilization, and in only a very minor population of cells. These data indicate that the human-specific down-regulation of Siglec-5 expression occurs at a pre-translational level. Given that the human-specific suppression of expression involves multiple CD33rSiglecs, these data are indicative of a mechanism of general transcriptional repression of the CD33rSiglec cluster.

The data provided herein indicate that a human-specific suppression of CD33rSiglec expression on T and B-cells occurred at some time prior to the emergence of modern humans ˜100-200,000 years ago. In keeping with this, the histogram of human T-cell responses to increasing TCR stimulation is markedly “shifted to the left” in comparison with chimpanzee T-cells. The data indicates that this activity contributes to an intrinsic hyper-reactivity of human T-cells, and may help explain the frequency and severity of T-cell-mediated diseases in the human species. In this regard, chimpanzee Siglec-5 was found to be particularly abundant on CD4+ T-cells. Such T-cells are involved in the pathology of many human diseases, including AIDS, chronic active hepatitis, inflammatory bowel disease, rheumatoid arthritis, type 1 diabetes, multiple sclerosis, psoriasis, etc. The lack of CD33rSiglec expression in humans may contribute to CD4 T-cell hyperactivity in these diseases. This may also help explain the unexpected interruption of a recent clinical trial in which healthy human volunteers became severely ill upon receiving an anti-CD28 mAb capable of directly stimulating T-cell activation (Wadman, (2006) Nature 440:388-389). The antibody had been previously tested in monkeys at concentrations much higher than those used in the humans, without significant adverse effects. The uniquely-human lack of CD33rSiglecs on T-cells may have allowed a marked stimulation of these cells in the subjects, perhaps releasing a “cytokine storm”.

CD33rSiglec expression differences on human and great ape B-cells also deserve further study. The presence of Siglecs in addition to CD22 may provide more stringent regulation of activation and function in chimpanzee cells compared to humans. In this regard, antibody self-reactivity related to disease (e.g., systemic lupus erythematosis or even a positive lupus antibody test) has not so far been reported in chimpanzees. It remains to be determined if CD33rSiglec expression down-regulates chimpanzee B-cell activation in a manner similar to CD22.

Activation of T-cells can also lead to cell death via apoptosis. The present data provides an explaination for increased T-cell activation and death observed in HIV-infected humans but not chimpanzees. Host proteins such as APOBEC3G and Trim5α are known to differ between old world monkeys and humans, helping explain species-specific susceptibility to HIV or SIV. However, such differences are not as prominent between humans and chimpanzees. Specifically, the critical amino acid at position 128 of APOBEC3G that confers African green monkey and rhesus macaque resistance to HIV and human resistance to SIV is not different between human and chimpanzees (33, 34). Furthermore, the human Trim5α sequence bears much greater similarity to chimpanzees (98% identity) than to rhesus macaques (87% identity). In addition, chimpanzee cells can be effectively infected by HIV, and the only major difference is the lack of severe CD4 attrition at later stages of the infectious process in vivo. Thus, the lack of progression to AIDS in chimpanzees may be due to the difference in responsiveness of the CD4 cells in general, the overall inflammatory condition during virus infection, and the reduced rate of proliferation and apoptosis of infected CD4 T-cells.

As with many events during evolution, it is difficult to be certain why humans are the only hominids without prominent expression of CD33rSiglecs on T-cells. The present studies indicate that this is not due to internal sequestration, but more likely to promoter- and/or transcription factor-mediated down-regulation of gene expression specific to human T-cells. Another possibility could be epigenetic changes affecting the CD33rSiglec cluster in humans. Early humans may have required a higher level of T-cell activation to defeat one or more pathogens, which was accomplished by down-regulating CD33rSiglecs. While this may have provided a short-term advantage, the long-term consequences may be the various T-cell-mediated diseases in humans today. Alternatively, CD33rSiglec loss from human T-cells could have occurred in the absence of pathogen pressure and the phenotype propagated in the small early human populations by random chance. In this regard, it is of note that most of the T-cell-mediated diseases mentioned occur in adults after the age of reproductive maturity, when selection forces are weak. The trait could thus be passed on without deleterious fitness effects on its carriers until recently, when human average lifespan increased. A third possibility arises from the human-specific loss of the Sia Neu5Gc, ˜3 million years ago. Possibly because of this dramatic change in human Sia biology, multiple human CD33rSiglecs appear to have undergone dramatic changes in other systems, involving gene deletion, gene conversion, and/or changes in binding specificity or expression (3, 4, 9-11). Thus, a possible side effect of this human-specific “shake-up” in Sia and CD33rSiglec biology was the almost complete loss of expression of the latter in T-cells. The expression of CD33rSiglecs on human T-cells has significantly diverged from that of other hominids.

Cells and Reagents: Great ape blood samples were collected into EDTA-containing tubes at the San Diego Zoo (San Diego, Calif.), the Yerkes National Primate Research Center (Atlanta, Ga.), or the Lincoln Park Zoo (Chicago, Ill.), and shipped on ice to UCSD. Human blood was collected from healthy volunteer donors, with approval from the UCSD IRB. These were collected at about the same time at UCSD and stored on ice, to ensure comparability in handling with the shipped great ape samples. Whole leukocyte preparations were isolated by ACK buffer (0.15 M NH4Cl, 10 mM KHCO3, 0.1 mM EDTA) lysis of RBCs, or PBMCs were isolated by centrifugation over Ficoll-Paque PLUS (Amersham Bioscience). Jurkat T-cell leukemia clone E6.1 cells from the American Type Culture Collection were cultured in RPMI-1640 supplemented with 10% FCS (cRPMI). Monoclonal antibodies against Siglec-6 (E20-1232), Siglec-7 (F023-420) and Siglec-9 (clone E10-286) were prepared in collaboration with BD Pharmingen, San Diego, Calif. The following antibodies were generously provided by Dr. Paul Crocker, University of Dundee, Scotland: anti-Siglec-5 (clone 1A5), anti-Siglec-7 (clones 7.5A and 7.7A), anti-Siglec-8 (clone 7C9), and anti-Siglec-10 (clone 5G6). Purified anti-CD33 (clone HIM3-4), anti-CD3 (clone UCHT-1), and anti-CD28 (clone CD28.2) were purchased from BD Pharmingen. R-phycoerythrin (PE) goat anti-mouse IgG (H+L) was purchased from Caltag Laboratories (Burlingame, Calif.). The plasmid construct pSig5 containing Siglec-5 under control of the CMV promoter was generated by cloning the full-length Siglec-5 cDNA into the multiple cloning site of pcDNA3.1(+) (Invitrogen, Carlsbad, Calif.).

Flow Cytometry: Cells (1×10⁶) were incubated with 1:100 dilution of antibody supernatant or 1 ug/100 ul purified Ab in 1% BSA in PBS for 30-60 min on ice. Cells, washed with 1% BSA in PBS, and resuspended in 100 ul of 1 ug/100 ul PE goat anti-mouse IgG conjugate in 1% BSA in PBS. For some experiments, cells were also labeled with allophycocyanin (APC)-anti-CD3, APC-anti-CD19, FITC-anti-CD4, or APC-anti-CD8 conjugates. Labeled cells were analyzed on a FACSCalibur (BD Biosciences) flow cytometer using CellQuest software. Data are presented using FlowJo software (Tree Star Inc.).

T-cell activation: Isolated human and chimpanzee PBMCs were cultured in RPMI with 5% human AB serum (RPMI-5HS). For plate-bound antibody-mediated stimulation, cells (2×10⁶/ml) were added to wells of a 12-well plate coated with 2.5 ug/ml anti-CD3. Anti-CD28 was then added to cells in solution at 0.1 ug/ml. For some experiments, anti-Siglec-5 was added at 1 ug/ml. Cells were cultured for 5 days, before being transferred to tubes and counted by flow cytometry at 60 ul/min for 30 seconds or for a maximum of 1×10⁶ cells. PBMCs were also stimulated with equal amounts of anti-CD3 and anti-CD28 in solution (0.04 to 1.0 μg/ml) or with 10 ug/ml of PHA (Sigma-Aldrich, St. Louis, Mo.) in solution for 3 to 5 days. Lymphocytes were also stimulated with anti-CD3/anti-CD28-bearing beads (DynabeadsO CD3/CD28 T-cell Expander, 4.5 μm, Dynal Biotech, Brown Deer, Wis.).

T-cell transfection: PBMCs were monocyte-depleted by incubation in a polystyrene T175 tissue culture flask at 1-2×10⁶/ml in RPMI-5HS for 1 h. Non-adherenT-cells were removed into a separate flask and confirmed to be mostly lymphocytes by flow cytometry. Lymphocytes or Jurkat T-cells were transfected using the Amaxa nucleofection technology™ (Amaxa Inc., Gaithersburg, Md.). Lymphocytes were resuspended with the Human T-cell Nucleofector Kit, while Jurkat T-cells were resuspended in Nucleofector Kit V, following the Amaxa guidelines for cell line transfection (see literature for details). Briefly, 100 ul of 2-5×10⁶ cell suspension mixed with 1-3 ug plasmid DNA (pSig5) was transferred to the provided cuvette and nucleofected with an Amaxa Nucleofector apparatus (Amaxa). Lymphocytes were transfected using the U-14 program and Jurkat T-cells with the S-18 program. Controls were mock-transfected using the same conditions with no DNA. Cells were immediately transferred into wells containing 37° C. pre-warmed culture medium in 12-well plates. After transfection, cells were cultured for 24 h before analysis by flow cytometry.

Intracellular calcium mobilization assay. Mobilization of intracellular calcium was measured using a real-time flow cytometric assay. Briefly, Fluo-4, AM (1 mM) and Fura Red (1 mM) calcium-sensing dyes (Molecular Probes) were mixed with Pluronic F-127 solution (Molecular Probes) at a volume ratio of 1:2:3. The calcium-sensing dye solution (2.5 μl) was added to Jurkat T-cells (4×10⁶/200 μl PBS) and incubated at 37° C. for 45 min. Cells were then washed with PBS, resuspended in 1 ml of PBS, and allowed to rest at RT for 30 min before stimulation. For analysis, cells were acquired using the time parameter on the FACSCalibur and analyzed for FL1 and FL3 fluorescence. The cell flow rate was 60 ul/sec (100-200 cells/sec). Anti-CD3 (0.5 μg) was added 60 s after beginning cell acquisition. Cells were collected for a total of 512 s. Post-collection analysis was performed using FlowJo software. The ratio of FL1:FL3 was derived and plotted over time. Kinetic plots are expressed as median of the FL1:FL3 ratio, which has been smoothed based on moving average.

### EXAMPLE 2

Siglec-F is a CD33rSiglec prominently expressed on mature circulating mouse eosinophils, and on some myeloid precursors in bone marrow. It has a binding preference for α2-3-linked Sias, with the best known ligand being 6′sulfo-sialyl-Lewis X. Interestingly, this structure is also the preferred ligand for human Siglec-8, a molecule also specifically expressed on human eosinophils. Although mouse Siglec-F is not the true ortholog of human Siglec-8, their marked similarities in expression patterns and ligand preferences indicate that they play equivalent roles. Studying Siglec-F in a mouse model should therefore provide insights into the currently unknown biological roles of typical CD33rSiglecs with ITIMs, as well as about the physiological functions of Siglec-8 in human eosinophils, and in eosinophil-mediated diseases.

The elevated eosinophil count in allergic conditions is well known, as is a critical role for CD4+ Th2 cells in regulating allergic inflammatory responses involving eosinophils. The data provided below elucidates the biological roles of Siglec-F in vivo. Data were generated using wild-type (WT) and Siglec-F null mice in an induced lung allergic response model associated with blood and bone marrow eosinophilia, tissue eosinophil accumulation and mediator release. This model also mimics some other features of bronchial asthma in humans, such as IgE-mediated mast cell activation and degranulation, airway inflammation and hyper-reactivity, CD4+ T-cell infiltration and cytokine production, goblet cell hyperplasia and mucus over-production. The studies described below with WT mice using this model indicate a negative feedback loop involving Siglec-F in controlling eosinophilic responses. This was confirmed by studies of Siglec-F null mice. These results represent the first demonstration of an in vivo biological role for a CD33rSiglec, and also reveal an unexpected role for CD33rSiglecs in regulating T-cell induction of eosinophilic responses.

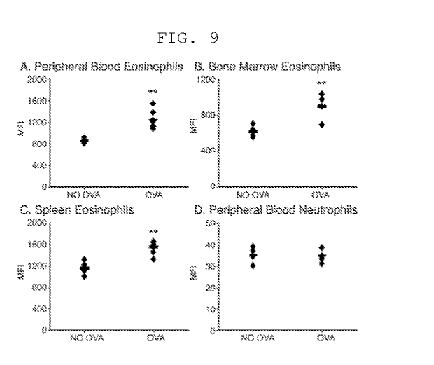

In order to evaluate the role of Siglec-F on eosinophils and in eosinophil-mediated diseases, a murine asthma-like lung allergy model was used in which eosinophils are recruited from the bone marrow to the lung. Mice were sensitized by repeated intraperitoneal injection of chicken ovalbumin (OVA), followed by intra-nasal challenge with the same antigen. Notably, eosinophils from blood, bone marrow and the spleen showed significantly increased Siglec-F levels after OVA challenge (FIG. 9, panels A-C). In contrast, neutrophils did not show any increase in their low levels of Siglec-F (FIG. 1, panel D, note that the Y axis scale is 100-fold lower for neutrophils).

Tissue sections of lungs of unchallenged mice showed very little staining with anti-Siglec-F antibodies. In contrast, staining of the inflamed lungs from the OVA challenged mice showed a marked infiltration with Siglec-F positive cells, especially in the peribronchial areas (FIG. 2, panel A). This staining overlapped with that for major basic protein (MBP), a specific eosinophil marker (FIG. 2, panel A).

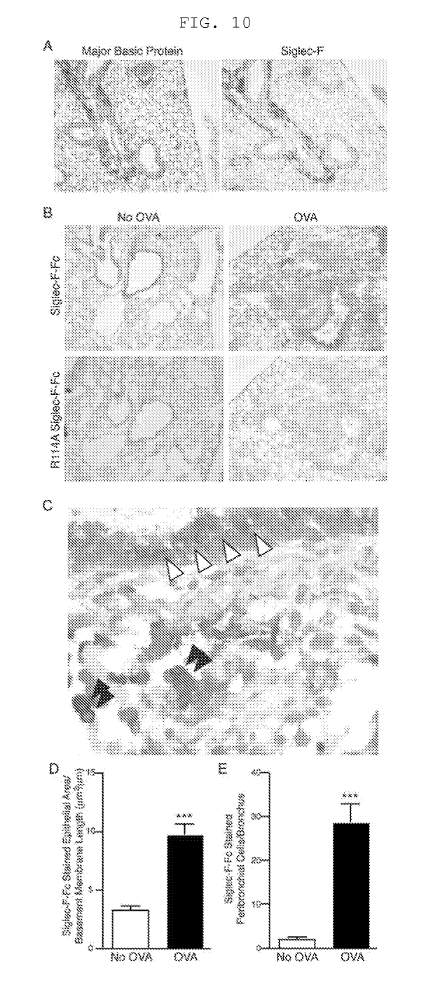

Sialic acid-dependent Siglec-F ligands are constitutively present in bronchial epithelia, and upregulated upon OVA challenge. The present studies also investigated the role of Siglec-F interactions with its ligands in normal and allergic conditions. The presence of Siglec-F ligands in the lung were investigated by probing tissue sections with Siglec-F-Fc, a recombinant soluble protein containing the extracellular domain of Siglec-F and the Fc region of human IgG (FIG. 10, panel B and panel C). In non-challenged mice, staining was detected only along the lining of the bronchial epithelium. Much reduced staining was observed with a mutant probe (R114A Siglec-F-Fc) deficient in Sia binding, confirming that binding of Siglec-F-Fc is indeed primarily Sia-dependent (FIG. 10, panel B). Interestingly, ligands were detected after OVA challenge not only on the bronchial epithelia (where it was increased in extent and amount; see e.g., FIG. 10, panel D), but also throughout the inflamed peribronchial area, that included mononuclear leukocytes (FIG. 10, panel E). No such upregulation of ligands was seen in non-OVA-sensitized mice given the intranasal antigen challenge only. Previous studies have shown that antibody-mediated cross-linking of CD33-related Siglecs cause negative signaling, and by analogy a natural ligand-mediated cross-linking likely causes a similar type of negative signaling. Taken together with the upregulation of Siglec-F on eosinophils upon OVA-challenge, these data indicate that Siglec-F and its ligands mediate a negative feedback loop controlling eosinophil-mediated allergic responses.

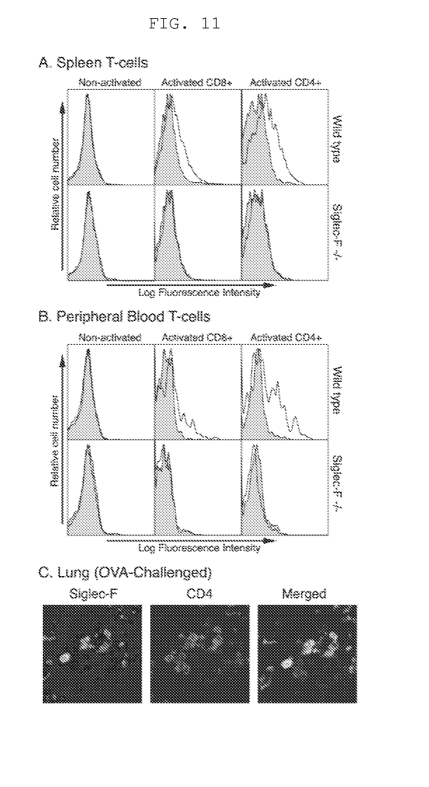

The present studies also show that Siglec-F expression is inducible on T-cells. Th2 cells also play a crucial role in allergic conditions. Since mouse T-cells are not known to express Siglecs, these cells were tested to determine if they express Siglec-F upon in vitro stimulation. Though non-activated T-cells did not express Siglec-F, both CD8+ and CD4+ T-cells from spleen and peripheral blood showed expression upon activation. Cells from Siglec-F-null mice (see corresponding information below) were used as a negative control, to confirm that all staining seen on the activated wild type (WT) mouse cells was specific (FIG. 11, panel A and panel B).

The present studies also determined whether the same induction occurs on CD4+ T-cells within the lung during an OVA-elicited allergic response. As there are very few peribronchial T-cells present during an acute OVA challenge, samples from mice were used that were OVA-challenged for a longer period of time—twice a week for one month, following regular acute challenge. Staining lung tissue sections from such mice with anti-CD4 and anti-Siglec-F antibodies showed some overlap of reactivity (FIG. 11, panel C), indicating induction of Siglec-F expression on some CD4+ T cells in vivo. Thus, induction of Siglec-F on CD4+ T-cells could be a further component of the proposed negative feedback loop regulating allergic responses in this model, in combination with the upregulation of Siglec-F ligands in the lung.

Also provided herein are novel Siglec-F-deficient mice. The data presented herein indicates that a lack of Siglec-F in a subject would allow an exaggerated eosinophilic response to OVA challenge. Siglec-F-null mice (hereafter called Siglec-F^{-/-} mice) were generated through homologous recombination and Cre-loxP-mediated excision of critical regions of the gene. While Siglec-F^{-/-} mice have a similar number of eosinophils as the WT controls, they lack Siglec-F expression.

Siglec-F^{-/-} mice were viable and fertile in a pathogen-free, limited-access barrier facility, with no obvious developmental or morphological defects. No abnormalities were found in baseline total blood cell counts, platelet counts, and blood chemistries. Leukocyte sub-group counts in lymphoid organs and serum immunoglobulin levels showed no changes, and the null mice were normal by histology studies. No differences were found between WT and Siglec-F^{-/-} mice in some immunological assays, such as air pouch-lipopolysaccharide inflammation looking at neutrophil recruitment, group A streptococcus skin infection and wound healing assays evaluating lesion formation and bacteria killing, or oxazolone-ear painting to test contact hypersensitivity. Overall, the Siglec-F-deficient mice are grossly normal in organ/tissue development and function, and in some innate immune responses not involving eosinophils.

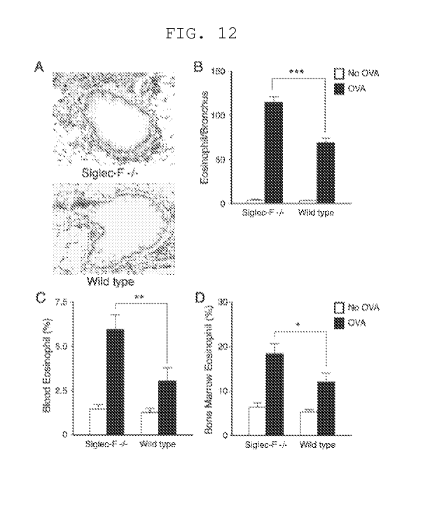

Siglec-F^{-/-} mice show enhanced lung eosinophilic inflammation, and peripheral blood and bone marrow eosinophilia in a lung allergy model. To test if Siglec-F plays a role in eosinophil-mediated disorders, Siglec-F^{-/-} and WT mice were compared in the lung responses to OVA challenge. OVA-challenged Siglec-F^{-/-} mice exhibited more prominent peribronchial eosinophil infiltration than the WT controls (FIG. 12, pane A and panel B). Eosinophils were enumerated in Wright-Giemsa stained bone marrow and peripheral blood smears. Although baseline levels were similar, the OVA-challenged Siglec-F^{-/-} mice had significantly more eosinophils in both blood and bone marrow (FIG. 12, panel C and panel D). In studying the bone marrow, an increase in metamyelocytic eosinophil precursors in OVA-challenged Siglec-F^{-/-} mice was identified. Thus, although Siglec-F^{-/-} mice have normal eosinophil levels in the baseline state, they manifest eosinophil over-production upon OVA challenge. These data indicate that the loss of Siglec-F expression accounts for the increased airway eosinophilia. There was no overall increase in numbers of infiltrating CD4+ cells in the lung tissues of the Siglec-F^{-/-} mice compared with WT mice.

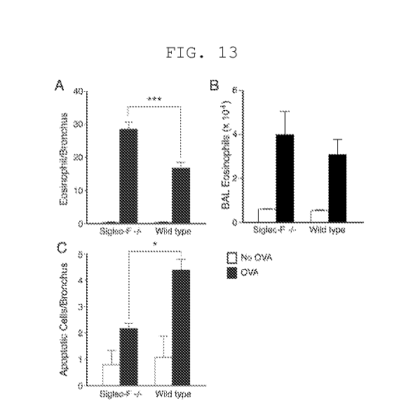

The present studies also show that eosinophil resolution is delayed, and apoptosis of peribronchial cells is impaired by Siglec-F deficiency. Eosinophil clearance or emigration from the lung was affected by Siglec-F deficiency. Siglec-F^{-/-} mice showed delayed eosinophil clearance from the lung (data from day 7 is presented in FIG. 13, panel A). There was no statistically significant change in BAL fluid eosinophil counts in the Siglec-F^{-/-} mice compared with that in WT mice (FIG. 13, panel B), indicating that the emigration of eosinophils is not significantly affected.

Human Siglec-8 can induce eosinophil apoptosis upon in vitro antibody cross-linking. The present studies have also determined that Siglec-F contributes to eosinophil apoptosis. TUNEL staining on lung sections revealed diminished peribronchial cell apoptosis in Siglec-F^{-/-} mice (FIG. 13, panel C). These apoptotic cells included eosinophils. Diminished eosinophil apoptosis in Siglec-F^{-/-} mice identifies at least one mechanism by which Siglec-F modulates eosinophil accumulation and helps explain the elevated peribronchial eosinophil accumulation and delayed eosinophil resolution.

In support of a role for Siglec-F in apoptosis, anti-Siglec-F antibody were found to induce enhanced apoptosis in eosinophils from IL-5 transgenic mice in vitro. Since it is difficult to study the proposed cross-linking of Siglec-F by the upregulated ligands in vitro, the effects of an anti-Siglec-F monoclonal antibody were examined. As it is difficult to obtain eosinophils from normal mice, cells from IL-5 transgenic mice were used. The data indicates that, while the antibody alone did not have any effect on the level of background apoptosis due to the withdrawal of IL-5, the addition of a secondary cross-linking antibody enhanced apoptosis.

Additional data provided herein indicates that Siglec-F elimination effects other features of the asthma-like response in mice. The OVA-challenge model in mice also shows some other features of classical bronchial asthma. Mucus production was evaluated using the traditional periodic acid Schiff staining to detect mucus-producing goblet cells. Overall, no clear difference was detected between Siglec-F^{-/-} and WT mice. To verify this result, and to explore an improved method to study mucus production, mucin sialic acid content in BAL was quantitated. This method takes advantage of the fact that mucins are resistant to proteinase digestion due to heavy O-glycosylation, allowing direct measurement of sialic acid content in the mucins. Using this method, a trend towards increased mucin expression in Siglec-F^{-/-} mice was identified (1.24±0.23 μM versus 1.76±0.20 μM sialic acid).

Some other typical features of classical asthma were also not obviously affected by Siglec-F deficiency under the conditions of this study. Total serum IgE levels were similar in OVA-challenged WT and Siglec-F^{-/-} mice. As mentioned earlier, there was no statistically significant change in BAL eosinophil number, and the number of peribronchial CD4+ cells was also unaffected by the Siglec-F deficiency. Airway hyper-responsiveness to methacholine aerosol was also check. Again, although a trend was noticed towards higher airway resistance in the Siglec-F^{-/-} mice, there was no overall significant difference detected in either invasive or non-invasive measurements. Regardless, the data with Siglec-F null mice in this lung allergy model indicate that Siglec-F and its ligands are upregulated as part of a negative feedback loop regulating eosinophilic and/or T-cell responses in allergy.

In conclusion, the induction of allergic lung inflammation in mice caused up-regulation of Siglec-F on blood and bone marrow eosinophils, accompanied by newly-induced expression on some CD4+ cells, as well as quantitative up-regulation of endogenous Siglec-F ligands in the lung tissue and airways. Taken together with the tyrosine-based inhibitory motif in the cytosolic tail of Siglec-F, the data indiactes a negative feedback loop, controlling allergic responses of eosinophils and helper T-cells, via Siglec-F and Siglec-F ligands. Allergen-challenged Siglec-F-null mice showed increased lung eosinophil infiltration, enhanced bone marrow and blood eosinophilia, delayed resolution of lung eosinophilia, and reduced peribronchial cell apoptosis. Anti-Siglec-F antibody cross-linking also enhanced eosinophil apoptosis in vitro. These data indicate a negative feedback role for Siglec-F, and represent the first in vivo demonstration of biological functions for any CD33rSiglec. These data further indicate that human Siglec-8 (the isofunctional paralog of mouse Siglec-F) can regulate the pathogenesis of human eosinophil-mediated disorders.

Provided herein is the first in vivo evidence for an inhibitory function of a CD33rSiglec. Expression of Siglec-F was upregulated on eosinophils and induced on T cells during an induced lung allergic response. Also, Sia-dependent ligands for Siglec-F were expressed in the lung airways, and upregulated during the allergic response. Studies in Siglec-F null mice confirmed involvement of the molecule in regulating eosinophil numbers. In this regard, it is notable that the Siglec-F null animals did not show any obvious eosinophil changes in their baseline state.

The delayed eosinophil clearance from the lung in Siglec-F^{-/-} mice may be partly due to diminished cell apoptosis. Apoptosis is an important mechanism to clear accumulated eosinophils and resolve airway eosinophilic inflammation, and correlates with the clinical severity of asthma. In vitro antibody cross-linking of Siglec-8 on isolated human eosinophils is known to cause apoptosis, and observed enhanced apoptosis of mouse eosinophils by antibody cross-linking of Siglec-F in vitro. The present data indicates that extensive cross-linking of Siglec-F by its ligands induces apoptosis of eosinophils under inflammatory conditions.

In addition to the possibility of direct induction of apoptosis or inhibition of marrow precursors and/or mature eosinophils by Siglec-F, this molecule may regulate eosinophil production and recruitment by modifying Th2 cell functions, which are known to play an essential role in allergic disorders such as asthma. Th2 cytokines like IL-5 play an important role in bone marrow eosinophil production, as well as in preventing eosinophil apoptosis. Thus, Siglec-F-mediated inhibition of Th2 cell cytokine production in vivo may influence the number of eosinophils, independent of direct effects of Siglec-F on eosinophils. The results provided herein indicate that the absence of Siglec-F enhances IL-5 production by OVA-stimulated T cells in vitro.

Mucus production was not significantly changed by Siglec-F deficiency. Since a variety of inflammatory mediators can stimulate mucus secretion, the lack of significant change is likely due to mediators produced from other cell types that do not express Siglec-F. Airway hyper-responsiveness was also not significantly affected by Siglec-F deficiency. In the mouse strain background used here, airway inflammation is more prominent than airway hyper-responsiveness.

The management of human asthma and several other eosinophil-related disorders has traditionally relied on symptomatic therapy and broadly acting agents such as corticosteroids, which can also have multiple side effects. The current work identifies one of the endogenous down-regulating mechanisms in such disorders. The data provided herein indicates that administration of synthetic ligands that cross-link Siglec-F can alleviate eosinophil-mediated disorders by a Siglec-F-dependent mechanism, such as augmentation of eosinophil clearance and/or inhibition of IL-5 production and release from Th2 cells. As presented above, these studies indicate that the human isofunctional paralog Siglec-8 contributes similarly in human eosinophil-mediated disorders. Accordingly, the present studies have identified a novel approach to the therapy of human asthma and other eosinophil-related diseases.

For the above-described studies, C57BL/6 mice were kept in a pathogen-free, limited-access barrier facility. Siglec-F null mice were generated as described in the Supplemental Methods. Mice that are 8-10 week old were used in experimental protocols approved by the UCSD Institutional Animal Care and Use Committee.

Rabbit antibody against mouse major basic protein (MBP) was obtained. R-phycoerythrin (PE)-conjugated rat anti-mouse Siglec-F (clone E50-2440) was derived from a hybridoma clone prepared in collaboration with BD Biosciences Pharmingen. The following materials were obtained from the sources indicated: hamster anti-mouse CD3 (clone 145-2C11) and hamster anti-mouse CD28 (clone 37.51): BD Biosciences Pharmingen; TriColor conjugated anti-mouse CD4 (clone CT-CD4) and anti-mouse CD8a (clone 5H10), Caltag; rat anti-mouse CD4 (clone GK 1.5), Chemicon; fluorescein conjugated rat anti-mouse CCR3 (clone 83101), R&D Systems. PE- or fluorescein-conjugated rat IgG2a K isotype (clone R35-95) were from BD Biosciences Pharmingen and served as the isotype-matched control antibodies.

Induction of allergic airway inflammation in mice. Pulmonary eosinophilia in mice was induced as previously described in Broide et al (J Immunol. (1998) 161:7054-7062). In brief, mice were sensitized by intraperitoneal injections on days 0 and 12 with 50 μg of ovalbumin (OVA; grade V, Sigma) adsorbed to 1 mg of alum (Aldrich) in 200 μl of phosphate-buffered saline (PBS). Intranasal OVA challenges (20 μg of OVA in 50 μl of PBS) were administered on days 24, 26, and 28 under isoflurane anesthesia. A control group was sensitized with OVA and then challenged with PBS, in place of OVA. On day 29, 24 h after the last OVA challenge, mice were examined for airway responsiveness and airway inflammation. Cell counts in blood smears and tissues described below were done in a blinded fashion.

For the airway inflammation resolution assay, mice were sensitized and intranasally challenged with OVA as described above. One group of mice were examined one day after the last challenge, and another group seven days after the last challenge, to look for the extent of resolution of inflammation.

For eosinophil counts in peripheral blood, bronchoalveolar lavage, and bone marrow, peripheral blood was collected from mice by cardiac puncture into EDTA-containing tubes. Erythrocytes were lysed using a 1:10 solution of 100 mM potassium carbonate:1.5 M ammonium chloride. The remaining cells were resuspended in 1 ml PBS. Bronchoalveolar lavage (BAL) was collected by lavaging the lung with 1 ml of PBS via a tracheal catheter 31. BAL was centrifuged, supernatant was collected and frozen at −80° C., and cells were resuspended in 1 ml PBS. Bone marrow cells were flushed from femurs with 1 ml PBS, centrifuged, and resuspended in 1 ml PBS. Total leukocytes were counted using a hemocytometer. To perform differential cell counts, 200 μl resuspended BAL cells, peripheral blood leukocytes, or 20 μl bone marrow cell suspensions were cytospun onto microscope slides and air-dried. Slides were stained with Wright-Giemsa and differential cell counts were performed under a light microscope.

For lung tissue eosinophil counts, lungs were inflated with an intratracheal injection of 4% paraformaldehyde solution, left overnight at 4° C., and were then embedded in paraffin, using standard procedures. They were sectioned at 5 μm onto slides 31, deparaffinized and hydrated. Endogenous peroxidase activity was quenched in 3% H₂O₂/methanol for 10 minutes. Sections were digested with pepsin for 10 min at 37° C., rinsed, and blocked for 30 min in goat serum in PBS. Slides were incubated with rabbit anti-mouse MBP (1:500) overnight in a moist chamber at 4° C. VECTASTAIN Rabbit ABC kit and AEC (3-amino-9-ethyl carbazole) reagent (Vector Laboratories) were used to detect immunoreactivity. Sections were counterstained with hematoxylin and mounted with aqueous mounting media. Peribronchial eosinophil counts were taken under a light microscope and 8-10 bronchi/slide were counted.

For cell apoptosis assays, TUNEL assays were performed to detect apoptotic cells in lung sections with an ApopTag Plus Peroxidase In Situ Apoptosis Detection Kit (Intergen) following manufacturer's instruction. After Methyl green (Vector Laboratories) counterstaining, apoptotic cells in the bronchus and peribronchial area were counted using light microscopy at 40× magnification. At least ten medium-sized bronchi were examined for each sample.

The effects of cross-linking an anti-Siglec-F antibody on mouse eosinophils was performed using mouse eosinophils purified from IL-5 transgenic mice (>95% purity) and incubated in media alone, or with anti-Siglec-F antibody (2.5 μg/ml), with or without a secondary cross-linking anti-rat IgG1/2a Ab (Pharmingen) (2.5 μg/ml) for 24 hours in a CO₂ incubator. The percentage of TUNEL-positive eosinophils was then determined on cytospin slide preparations.

In vitro activation of T cells was performed using mononuclear cells isolated from peripheral blood or spleen by Ficoll-Paque centrifugation, washed with PBS, and resuspended in RPMI 1640 media supplemented with 10% fetal bovine serum, 2 mM L-glutamine, 100 U/ml penicillin and 100 μg/ml streptomycin sulfate. The cells (0.5×10⁶/well) were transferred to 48-well plates pre-coated with anti-mouse CD3 (2 μg/well) and anti-mouse CD28 (1 μg/well), and cultured for 3 days.

Flow cytometric analysis of Siglec-F was performed using leukocytes from blood or spleen or in vitro-activated T-cells incubated with anti-mouse CD16/CD32 to block FcγIII/II receptor. Each sample (1×10⁵ cells) was then stained with anti-mouse Siglec-F-PE and anti-mouse CCR3-fluorescein, anti-mouse CD4-TriColor or anti-mouse CD8-TriColor, and subjected to flow cytometric analysis. FACSCalibur (BD Biosciences) and Flowjo software (Tree Star) were used to collect and analyze the data.

Detection of Siglec-F and CD4 in lung sections was performed using paraffin-embedded lung sections rehydrated, quenched with 3% H₂O₂/methanol for 1 h, and subjected to antigen retrieval (5 min×2 in a microwave). Sections were then blocked and immunostained with rat anti-mouse Siglec-F (1:20) at 4° C. overnight, followed by biotinylated goat anti-rat IgG (1:200) for 1 h, and peroxidase-conjugated streptavidin (1:100) for 1 h. Using a TSA kit (Molecular Probes), slides were incubated with Tyramide-Alexa555 in 0.0015% H₂O₂ for 10 min. Slides were washed and blocked again. CD4 staining was performed with rat anti-mouse CD4 (1:1,000) at 4° C. overnight, followed by biotinylated goat anti-rat IgG (1:200) for 1 h, and streptavidin-dichlorotriazinylamino fluorescein (1:300; Jackson ImmunoResearch Laboratories) for 1 h. Sections were scanned using a Nikon Eclipse E800 microscope (Nikon). Images were captured and analyzed using Microfire (Olympus America, Karl Storz Imaging). Images were overlaid using Photoshop software.

In order to probe lung sections for Siglec-F ligands, cryostat sections of lung tissues were air-dried, fixed in acetone for 10 min, quenched in 0.03% H₂O₂/methanol for 30 min, and blocked for endogenous biotin. Slides were incubated with recombinant Siglec-F-Fc or R114A Siglec-F-Fc, followed by biotin-conjugated goat anti-human IgG (1:750; Vector Laboratories) for 30 min, peroxidase-conjugated streptavidin (1:500; Jackson ImmunoResearch Laboratories) for 30 min, and Vector NovaRed (Vector Laboratories) for 40 min. Slides were counter-stained with Mayer's hematoxylin.

Quantitative analysis of Siglec-F-Fc immunostained epithelium and peribronchial cells was also performed. Lungs from OVA and non-OVA challenged WT mice (n=2 each) were used for Siglec-F-Fc immunostaining as above, and subjected to quantitative image analysis. Following lung immunostaining, the area of epithelial Siglec-F-Fc immunostaining was outlined and quantified using a light microscope (Leica DMLS: Leica Microsystems Inc., Depew, N.Y., USA) attached to an image-analysis system (Image-Pro Plus: MediaCybernetics, Silver Spring, Md., USA). Results are expressed as the area of epithelial immunostaining per μm length of epithelial basement membrane of bronchioles with 150-250 μm internal diameter. The number of individual non-epithelial cells in the peribronchial space that immunostained positive for Siglec-F-Fc were also counted using a light microscope. Results are expressed as the number of peribronchial cells immunostained per bronchiole. At least ten bronchioles were counted in each slide.

The evaluation of BAL mucin production was performed by Sia quantification. Briefly, BAL (20 μl) was mixed well with methanol and chloroform at a 1:10:10 ratio, and centrifuged to extract lipids. The protein-containing pellet was air-dried, and mucin fragments isolated based on modification from the recent method for isolating carcinoma mucins.

Airway responsiveness to methacholine was assessed 24 h after the final OVA challenge in intubated and ventilated mice as described.

Results from the different groups were compared by two-tailed Student's t-test using a statistical software package (In Stat, GraphPad Software). All results are given as mean±standard error of the mean. P values of <0.05 were considered statistically significant.

The examples set forth above are provided to give those of ordinary skill in the art a complete disclosure and description of how to make and use the embodiments of the apparatus, systems and methods of the invention, and are not intended to limit the scope of what the inventors regard as their invention. Modifications of the above-described modes for carrying out the invention that are obvious to persons of skill in the art are intended to be within the scope of the following claims. All patents and publications mentioned in the specification are indicative of the levels of skill of those skilled in the art to which the invention pertains. All references cited in this disclosure are incorporated by reference to the same extent as if each reference had been incorporated by reference in its entirety individually.

A number of embodiments of the invention have been described. Nevertheless, it will be understood that various modifications may be made without departing from the spirit and scope of the invention. Accordingly, other embodiments are within the scope of the following claims.

What is claimed is:

1. A method for modulating lymphocyte activation, the method comprising contacting a lymphocyte with an agent that increases the activity of a target Sialic acid-recognizing Ig-superfamily lectin (Siglec) associated with the lymphocyte.

2. The method of claim 1, wherein the lymphocyte is a T-cell.

3. The method of claim 2, wherein the T-cell is a CD4 T-cell.

4. The method of claim 1, wherein the lymphocyte is a B-cell.

5. The method of claim 1, wherein the target Siglec is a CD33 related Siglec (CD33rSiglec).

6. The method of claim 5, wherein the CD33 related Siglec is selected from the group consisting of Siglec-2, Siglec-3, Siglec-5, Siglec-6, Siglec-7, Siglec-8, Siglec-9, Siglec-10, Siglec-11, Siglec-12, Siglec-14, and homologs thereof.

7. The method of claim 1, wherein the modulating is inhibition of lymphocyte activation.

8. The method of claim 1, wherein lymphocyte activation is lymphocyte proliferation.

9. The method of claim 1, wherein increasing the activity of a target Siglec comprises increasing the expression of endogenously-produced target Siglec.

10. The method of claim 1, wherein increasing the activity of a target Siglec comprises expressing recombinantly-produced target Siglec.

11. The method of claim 1, wherein increasing the activity of a target Siglec comprises increasing stability of endogenously-produced target Siglec.

12. A method for treating a subject having, or susceptible to having, a lymphocyte-mediated pathology, the method comprising administering to the subject an agent that modifies the activity of a target Siglec associated with a B-lymphocyte or T-lymphocyte.

13. The method of claim 12, wherein the lymphocyte-mediated pathology is selected from the group consisting of rheumatoid arthritis, chronic active hepatitis asthma, inflammatory bowel disease (IBD), multiple sclerosis (MS), psoriasis, toxic shock syndrome, HIV progression to AIDS and Systemic lupus erythematosus (SLE).

14. The method of claim 12, wherein the lymphocyte is a T-cell.

15. The method of claim 14, wherein the T-cell is a CD4 T-cell.

16. The method of claim 12, wherein the lymphocyte is a B-cell.

17. The method of claim 12, wherein the target Siglec is a CD33 related Siglec (CD33rSiglec).

18. The method of claim 17, wherein the CD33 related Siglec is selected from the group consisting of Siglec-2, Siglec-3, Siglec-5, Siglec-6, Siglec-7, Siglec-8, Siglec-9, Siglec-10, Siglec-11, Siglec-12, Siglec-14, and homologs thereof.
